(19) United States
(12) Patent Application Publication
(10) Pub. No.: US 2012/0282276 A1
(43) Pub. Date: Nov. 8, 2012

(54) BIOMARKERS PREDICTIVE OF PROGRESSION OF FIBROSIS

(75) Inventors: Cory Hogaboam, Ann Arbor, MI (US); Steven L. Kunkel, Ann Arbor, MI (US); Glenda Trujillo, Ann Arbor, MI (US)

(73) Assignees: THE REGENTS OF THE UNIVERSITY OF MICHIGAN, Ann Arbor, MI (US); NOVARTIS AG, Basel (CH)

(21) Appl. No.: 13/505,985

(22) PCT Filed: Nov. 4, 2010
(86) PCT No.: PCT/EP2010/066786

§ 371 Date: Jul. 23, 2012

Related U.S. Application Data
(60) Provisional application No. 61/258,293, filed on Nov. 5, 2009.

(51) Int. Cl. C12Q 1/68; C40B 30/06; C12N 5/071; C12N 5/077; A61P 1/16; A61K 31/711; A61K 38/16; A61P 11/00; A61P 7/00; G01N 33/566; A61K 39/395

(52) U.S. Cl. 424/172.1; 435/6.12; 435/6.11; 435/6.17; 435/7.21; 506/10; 435/375; 514/44 R; 514/1.1; 514/1.8

(57) ABSTRACT

The present invention provides methods and kits for prognosing the progression of fibrosis in a subject having fibrosis, as well as methods for identifying a compound that can slow down the progression of fibrosis in a subject having fibrosis, methods of monitoring the effectiveness of a therapy in reducing the progression of fibrosis in a subject having fibrosis, methods of selecting a subject for participation in a clinical trial for the treatment of fibrosis, and methods for inhibiting progression of fibrosis in a cell or a subject having fibrosis. The methods are based on determining the level of Toll-like recepter 9 (TLR9).

### RELATED APPLICATIONS

This application claims priority to U.S. Provisional Patent Application Ser. No. 61/258293, filed on Nov. 5, 2009. The entire contents of the foregoing application are incorporated herein by reference.

### BACKGROUND OF THE INVENTION

Fibrosis is the formation of excessive fibrous tissue. Fibrosis may be the result of response to necrosis, injury, or chronic inflammation, which may be induced by a wide variety of agents, e.g., drugs, toxins, radiation, any process disturbing tissue or cellular homeostasis, toxic injury, altered blood flow, infections (viral, bacterial, spirochetal, and parasitic), storage disorders, and disorders resulting in the accumulation of toxic metabolites. Fibrosis is most common in the heart, lung, peritoneum, and kidney.

One type of fibrosis of the lung is idiopathic pulmonary fibrosis (IPF). IPF is a chronic, generally progressive lung disease with high mortality and unmet clinical needs. It is widely accepted that IPF transpires from an unknown insult to the lung that leads to irreversible scarring marked by severe alveolar destruction, variable inflammation accompanied by excessive deposition of extracellular matrix, and ultimate loss of normal lung function (Wynn, T. A. (2008) J Pathol 214:199-210). The pathogenesis of IPF is not completely understood, although persistent fibroblast proliferation and activation remains at the forefront of targetable mechanisms for the therapeutic intervention of IPF. Fibroblasts are fundamental to homeostasis and normal wound repair through the production of extracellular matrix (ECM) proteins. In fibrotic diseases, the unregulated proliferation of fibroblasts, their differentiation into myofibroblasts, and the excessive production of ECM leads to destruction of normal interstitial architecture.

Increasingly, it has become evident that the disease course in IPF patients is extremely variable with some patients exhibiting relative disease stability for prolonged periods of time while others exhibit rapid disease progression (Martinez, F. J., et al. (2005) Ann Intern Med 142:963-967). Although some IPF patients exhibit physiological decline others experience acute deterioration, acute exacerbation of IPF (AE-IPF) (Hyzy, R., et al. (2007) Chest 132, 1652-1658; Collard, H. R., et al. (2007) Am J Respir Crit Care Med 176:636-643). As such, increasingly disease progression in IPF patients has been defined using a composite approach which includes physiological progression, AE-IPF and/or all cause mortality. Rigorous studies aimed at understanding the etiology, risk factors, and pathogenesis of disease progression is required for the accurate management of IPF. As many current treatment studies emphasize intermediate term outcomes, defining disease course during an initial evaluation would have great practical value.

Thus, there is an urgent need in the field for better prognostic indicators for the progression of fibrosis in patients suffering from fibrosis, e.g., IPF, as well as for more effective methods for inhibiting the progression of fibrosis in patients suffering from fibrosis.

### SUMMARY OF THE INVENTION

The present invention provides methods and kits for prognosing the progression of fibrosis in a subject having fibrosis, as well as methods for identifying a compound that can slow down the progression of fibrosis in a subject having fibrosis, methods of monitoring the effectiveness of a therapy in reducing the progression of fibrosis in a subject having fibrosis, methods of selecting a subject for participation in a clinical trial for the treatment of fibrosis, and methods for inhibiting progression of fibrosis in a cell or a subject having fibrosis.

The present invention is based, at least in part, on the discovery that TLR9 is overexpressed in lung biopsies of IPF patients clinically classified as exhibiting rapid disease progression over the first year of follow-up. The present invention is also based, at least in part, on the discovery that TLR9 expression in lung fibroblasts is upregulated in vitro by unmethylated CpG DNA motifs present on bacterial and viral DNA. Using an adoptive transfer model with human lung fibroblasts from rapid or stable progressors into immunodeficient C.B.17SCID/bg mice, it was demonstrated that fibroblasts from rapid progressors cause increased fibrosis in the murine lung that is exacerbated when mice received a single intranasal CpG challenge. The data presented herein show for the first time that CpG induces the differentiation of human CD14+ monocytes into fibrocyte-like cells and mediates EMT in human A549 lung epithelial cells.

Accordingly, the present invention provides methods for predicting the progression of fibrosis in a subject having fibrosis. The methods include determining the level of expression of Toll-like receptor 9 (TLR9) in a sample from the subject; and comparing the level of expression of TLR9 in the sample from the subject to the level of expression of TLR9 in a control sample, wherein an increase in the level of expression of TLR9 in the sample from the subject as compared to the level of expression of TLR9 in the control sample is an indication that the fibrosis will rapidly progress, thereby predicting the progression of fibrosis in the subject having fibrosis.

In another aspect, the invention provides methods for identifying a compound that can slow down the progression of fibrosis in a subject having fibrosis. The methods include separately contacting an aliquot of a sample from the subject with each member of a library of compounds; determining the effect of a member of the library of compounds on the level of expression of Toll-like receptor 9 (TLR9) in each of the aliquots; and selecting a member of the library of compounds which decreases the level of expression of TLR9 in an aliquot as compared to the level of expression of TLR9 in a control sample, thereby identifying a compound that can slow down the progression of fibrosis in a subject having fibrosis.

In yet another aspect, the invention provides methods of monitoring the effectiveness of a therapy in reducing the progression of fibrosis in a subject having fibrosis. The methods include determining the level of expression of Toll-like receptor 9 (TLR9) in a sample from the subject prior to and following administration of at least a portion of the therapy to the subject; and comparing the level of expression of TLR9 in the sample from the subject prior to the administration of the therapy to the level of expression of TLR9 in the sample from the subject following administration of at least a portion of the therapy, wherein a decrease in the level of expression of TLR9 in the sample following administration of at least a portion of the therapy as compared to the level of expression of TLR9 in the sample prior to the administration of the therapy is an indication that the subject is responding to the therapy, thereby monitoring the effectiveness of the therapy in reducing the progression of fibrosis in the subject having fibrosis.

In another aspect, the invention provides methods of selecting a subject for participation in a clinical trial for a treatment of fibrosis. The methods include determining the level of expression of Toll-like receptor 9 (TLR9) in a sample from a subject having fibrosis, and comparing the level of expression of TLR9 in the sample from the subject to the level of expression of TLR9 in a control sample, wherein a higher level of expression of TLR9 in the sample from the subject as compared to the level of expression of TLR9 in the control sample is an indication that the subject should participate in the clinical trial, thereby selecting a subject for participation in a clinical trial for a treatment of fibrosis.

In one embodiment of the invention, the fibrosis is selected from the group consisting of idiopathic pulmonary fibrosis, liver fibrosis following liver transplantation, liver fibrosis following chronic hepatitis C virus infection, and interstitial fibrosis in focal segmental glomerulosclerosis. In another embodiment of the invention, the fibrosis is selected from the group consisting of cystic fibrosis of the pancreas and lungs, injection fibrosis, endomyocardial fibrosis, mediastinal fibrosis, myelofibrosis, retroperitoneal fibrosis, progressive massive fibrosis, nephrogenic systemic fibrosis. In one embodiment, the fibrosis is caused by surgical implantation of an artificial organ.

The methods of the invention may further comprise determining the presence or absence of unmethylated CpG in the sample from the subject; determining the presence or absence of a gammaherpesvirus in the sample from the subject; and/or determining the level of expression in the sample of an additional marker selected from the group consisting of annexin 1, alpha smooth muscle actin, neutrophil elastase, KL-6, ST2, IL-8, alpha defensin, beta3-endonexin, serine protease inhibitor, Kazal type, plasminogen activator inhibitor-1, HPS3, Rab38, Smad6, ADAMTS7, CXCR6, Bcl2-L-10, and MMP-9.

The level of expression of TLR9 in the sample may be determined by detecting the presence in the sample of a transcribed polynucleotide, or portion thereof, of TLR9 gene. The step of detecting may comprise the step of detecting cDNA and/or amplifying the transcribed polynucleotide. The level of expression of TLR9 in the sample may also be determined by detecting the presence in the sample of TLR9 protein by, for example, using an antibody, or antigen binding fragment thereof, which specifically binds to the protein.

The level of expression of TLR9 in the sample may be determined by using a technique selected from the group consisting of polymerase chain reaction (PCR) amplification reaction, reverse-transcriptase PCR analysis, quantitative reverse-transcriptase PCR analysis, Northern blot analysis, Western blot analysis, immunohistochemistry, ELISA assay, array analysis, and combinations or sub-combinations thereof.

The sample obtained from the subject may comprise a fluid, such as a fluid selected from the group consisting of fluids collected by bronchial lavage, blood fluids, vomit, intra-articular fluid, saliva, lymph, cystic fluid, urine, fluids collected by peritoneal rinsing, and gynecological fluids. In one embodiment, the sample from the subject is a fluid collected by bronchial lavage. The sample obtained from the subject may also or alternatively comprise a tissue, or component thereof, such as a tissue selected from the group consisting of lung, connective tissue, cartilage, lung, liver, kidney, muscle tissue, heart, pancreas, bone, and skin. In one embodiment, the tissue is lung, or a component thereof.

In one embodiment of the invention, the subject is human.

In another aspect, the invention provides kits for predicting the progression of fibrosis in a subject having fibrosis. The kits include means for determining the level of expression of Toll-like receptor 9 (TLR9) and instructions for use of the kit to predict the progression of fibrosis in the subject having fibrosis.

In another aspect, the present invention provides kits for predicting the progression of fibrosis in a subject having fibrosis. The kits include means for obtaining a biological sample from the subject, means for determining responsiveness of the sample to TGFβ and CpG, and instructions for use of the kit to predict the progression of fibrosis in the subject having fibrosis. In one embodiment, such kits further comprise means for determining the level of expression of Toll-like receptor 9 (TLR9). In another embodiment, such kits do not comprise means for determining the level of expression of TLR9.

In various embodiments, the kits of the invention may further comprise means for obtaining a biological sample from a subject; a control sample; means for determining the presence or absence of unmethylated CpG; means for determining the presence or absence of a gammaherpesvirus; and/or means for determining the level of expression of an additional marker selected from the group consisting of annexin 1, alpha smooth muscle actin, neutrophil elastase, KL-6, ST2, IL-8, alpha defensin, beta3-endonexin, serine protease inhibitor, Kazal type, plasminogen activator inhibitor-1, HPS3, Rab38, Smad6, ADAMTS7, CXCR6, Bcl2-L-10, and MMP-9.

In yet another aspect, the invention provides methods of inhibiting the progression of fibrosis in a cell, e.g., pulmonary cell, a liver cell, a kidney cell, a cardiac cell, a musculoskeletal cell, a skin cell, an eye cell, or a pancreatic cell. The methods include contacting the cell with an effective amount of a TLR9 antagonist, thereby inhibiting progression of fibrosis in the cell.

In another aspect, the invention provides methods for inhibiting the progression of fibrosis in a subject, e.g., a human subject, by administering an effective amount of a TLR9 antagonist to the subject, thereby inhibiting the progression of fibrosis in the subject. In one embodiment, such methods may further comprise administering to the subject an additional therapeutic agent.

In one embodiment, the antagonist is administered intravenously, intramuscularly, or subcutaneously to the subject.

In another embodiment, the fibrosis is selected from the group consisting of idiopathic pulmonary fibrosis, liver fibrosis following liver transplantation, liver fibrosis following chronic hepatitis C virus infection, and interstitial fibrosis in focal segmental glomerulosclerosis. In yet another embodiment of the invention, the fibrosis is selected from the group consisting of cystic fibrosis of the pancreas and lungs, injection fibrosis, endomyocardial fibrosis, mediastinal fibrosis, myelofibrosis, retroperitoneal fibrosis, progressive massive fibrosis, nephrogenic systemic fibrosis. In one embodiment, the fibrosis is caused by surgical implantation of an artificial organ.

In one embodiment, the TLR9 antagonist is selected from the group consisting of an antibody, e.g., a murine antibody, a human antibody, a humanized antibody, a bispecific antibody and a chimeric antibody, a Fab, Fab′2, ScFv, SMIP, affibody, avimer, versabody, nanobody, or a domain antibody; a small molecule; a nucleic acid, e.g, an antisense molecule, e.g., RNA interfering agent and a ribozyme; a fusion protein; an adnectin; an aptamer; an anticalin; a lipocalin; or TLR9-derived peptidic compound.

Other features and advantages of the invention will be apparent from the following detailed description, and from the claims.

### BRIEF DESCRIPTION OF THE DRAWINGS

FIGS. 1A-1D depict various clinical features of patients with rapid and slowly progressive forms of Idiopathic Pulmonary Fibrosis (IPF) and TLR9 expression. A. The survival of IPF patients classified as rapid or slow progressors. B. Representative histology of IPF in a patient with slow (1, 2) and rapid (3,4) progression shown at 20× and 40× magnification. C. Quantitative TaqMan PCR analysis of TLR9 gene expression in upper lobe SLBs from rapid and slow progressors. The data shown are the mean of all the combined upper lobe mRNA values compared to the mean of normal SLBs mRNA values (standardized to GAPDH housekeeping gene). The error bar shows the SEM of all the data in the rapid (n=10) and stable (n=13) progressor patient groups. The two-tailed P value was determined by the unpaired t test with Welch correction. D. Representative immunohistochemical staining of TLR9 in SLBs from a total of 7 slow (1) and 5 rapid (3) progressors shown at 20× magnification. Corresponding fields stained with isotype control (IgG) shown in 2 and 4.

FIGS. 2A-2F depict the induction of differentiation of CD14+ human into fibrocyte-like cell . A. Experimental scheme for the in vitro differentiation of CD14+ monocytes. B. Photomicrographs of monocytes cultured in serum-free media or serum-free media containing 10 ng/ml TGFβ and stimulated with nothing (1,2), 50 μg/mL non CpG (3,4), 50 μg/mL CpG (5,6), or 50 μg/mL poly IC (7,8) on Day 3. C. qRT-PCR analysis of fibrocyte markers. αSMA gene expression in monocytes cultured for 3 days in serum-free media +/−CpG for 24 hours (1). Collagen I gene expression in monocytes cultured for 3 days in serum-free media or TGFβ, +/−CpG or poly I:C (2). D. Fluorescent ICC for collagen I in monocytes (40× magnification) cultured in serum-free media (1) or TGFβ (2); serum-free media+CpG (3), or TGFβ+CpG (4). Isotope control for monocytes cultured in TGFβ+CpG (5). Representative (n=3) FC for collagen expression as percent of total CD14+CD45+cells from monocytes cultured in serum-free media and serum-free media containing CpG E. Forward and side scatter FC of monocytes cultured in serum-free media containing TGFβ (1) or TGFβ+CpG (2). Representative (n=3) FC for CD14 as percent of total cells from monocytes cultured in serum-free media (3) or monocytes cultured in serum-free media containing TGFβ (4) stained with anti-CD 14. Representative (n=3) FC for CD45 as percent of CD 14− cells from monocytes cultured in serum-free media (5) or monocytes cultured in serum-free media containing TGFβ (6) stained with anti CD45 and gated with respect to CD14 expression. Representative data (n=3) is graphed as percent of CD14+ cells from monocytes cultured in serum-free media and monocytes cultured in serum-free media containing TGFβ stained with anti-CD45 and gated with respect to CD14 expression.

FIGS. 3A-3E depict epithelial-mesenchymal transition (EMT) in human A549 cells induced by CpG. A. Representative photomicrographs (n=5) of A549 cells cultured in media (DMEM+10% FCS) (1), TGFβ (2), and increasing CpG concentrations of 5 μg/mL (3), 10 μg/mL (4), 50 μg/mL (5), 100 μg/mL (6), and 200 μg/mL (7) for 96 hours. B. qRT-PCR analysis of aSMA (1), vimentin (2), and e-cadherin (3) in A549 cells cultured with increasing concentrations of CpG for 96 hours. C. qRT-PCR analysis of IFNa in A549 cells cultured with increasing concentrations of CpG for 96 h. D. Fluorescent ICC for collagen 1 in A549 cells (40× magnification) that were cultured for 96 hours in media (1), 10 μg/mL CpG (2), 50 μg/mL CpG (3), and 100 μg/mL CpG (4). Isotope control for collagen I antibody using cells cultured with 100 μg/mL CpG (5). E. siRNA knockdown of TLR9 in A549 cells in a CpG EMT assay: Western Blot analysis of TLR9 protein and β-actin loading control in A549 cell lysates after siRNA treatment with a non targeting control siRNAs, cyclophilin B control siRNAs , and TLR9 siRNAs; (1-4) photomicrographs of A549 cells before CpGDNA treatment cultured in media and transfection agent alone (5), with non target siRNA (6) , and with TLR9 siRNA (7); representative photomicrographs (n=4) of A549 cells after siRNA treatment and stimulated with media and transfection agent alone (8), non target siRNA+75 μg/ml CpG (9), and TLR9 siRNA+75 CpG-DNA for 72 hrs (10) ; qRT-PCR analysis of vimentin (11) and e-cadherin (12) in siRNA-treated A549 cells and cultured with 75 μg/ml CpG for 72 hours. Data are mean±SD. ***p<0.0001.

FIGS. 4A-4J depict TLR9 expression in rapid and slowly progressive IPF lung fibroblasts and response to CpG. A. qRT-PCR analysis of TLR9 gene expression in representative rapid UIP/IPF(n=5-8) (a) and slow IPF (n=5-8) (b) fibroblast cell lines treated for 24 hour without (untreated) or with CpG-ODN (10 μg/mL) in the presence or absence of IL-4 (10 ng/ml). Fold increase is calculated within each group of disease compared with the respective untreated fibroblasts. Bioplex analyses of rapid or slow IPF fibroblast conditioned media for IFNα (c and d), PDGF (e and f), MCP-1/CCL2 (g and h), and MCP-3/CCL3 (i and j). Fibroblast cell lines were treated for 24 hours without (untreated) or with CpG-ODN (10 μg/mL) in presence or absence of IL-4 (10 ng/ml). Data is representative of at least 5 slow IPF and 5 rapid IPF fibroblast cell lines. Data are mean±SEM. **p<0.001 and ***p<0.0001.

FIGS. 5A-5C depict the exacerbation of fibrosis induced by CpG in rapidly progressive human lung fibroblasts in a human-SCID mouse model of IPF. A. Experimental scheme for establishing a human-SCID model of AE-IPF. B. Representative mouse lung sections stained with Masson's trichrome to depict degree of fibrosis from mice that received normal human lung fibroblasts and intranasally challenged on Day 35 with saline (1) or CpG (2), rapid UIP/IPF human lung fibroblasts intranasally challenged on Day 35 with saline (3) or CpG (4), and slow UIP/IPF human lung fibroblasts intranasally challenged on Day 35 with saline (5) or CpG (6). C. Hydroxyproline levels in half lung homogenates from saline-challenged or CpG-challenged mice that received rapid UIP/IPF human lung fibroblasts (1) and stable UIP/IPF human lung fibroblasts (2). Data are mean±SEM from five mice at each time point. Data are mean±SEM. **p<0.001.

### DETAILED DESCRIPTION OF THE INVENTION

The present invention is based, at least in part, on the discovery that TLR9 is overexpressed in lung biopsies of IPF patients clinically classified as exhibiting rapid disease progression over the first year of follow-up. The present invention is also based, at least in part, on the discovery that TLR9 expression in lung fibroblasts is upregulated in vitro by unmethylated CpG DNA motifs present on bacterial and viral DNA. Using an adoptive transfer model with human lung fibroblasts from rapid or stable progressors into immunodeficient C.B.17SCID/bg mice, it was demonstrated that fibroblasts from rapid progressors cause increased fibrosis in the murine lung that is exacerbated when mice received a single intranasal CpG challenge. The data presented herein show for the first time that CpG induces the differentiation of human CD 14+ monocytes into fibrocyte-like cells and mediates EMT in human A549 lung epithelial cells.

Accordingly, methods and kits are provided herein for prognosing the progression of fibrosis in a subject having fibrosis, as well as methods for identifying a compound that can slow down the progression of fibrosis in a subject having fibrosis, methods of monitoring the effectiveness of a therapy in reducing the progression of fibrosis in a subject having fibrosis, methods of selecting a subject for participation in a clinical trial for treatment of fibrosis, and methods for inhibiting progression of fibrosis in a cell or a subject having fibrosis.

Although the alteration of the level of expression of TLR9 described herein was identified in idiopathic pulmonary fibrosis (IPF), the methods of the invention are in no way limited to use for the prognosis, diagnosis, characterization, therapy and prevention of IPF, e.g., the methods of the invention may be applied to any fibrotic disease as described herein.

Various aspects of the invention are described in further detail in the following subsections:

### I. Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

As used herein, the term “fibrosis” refers to the aberrant formation or development of excess fibrous connective tissue in a cell, organ or tissue. Fibrosis occurs as part of a reparative or reactive process in a cell, tissue, or organ due to, for example, physical injury, inflammation, infection, and exposure to toxins. There are several types of fibrosis, for example, cystic fibrosis of the pancreas and lungs; injection fibrosis, which can occur as a complication of intramuscular injections, especially in children; endomyocardial fibrosis; pulmonary fibrosis of the lung; mediastinal fibrosis; myleofibrosis; retroperitoneal fibrosis; progressive massive fibrosis, a complication of coal workers' pneumoconiosis; and nephrogenic systemic fibrosis.

As used herein, the term “fibrosis” may be used interchangeably with the terms “fibrotic disorder”, “fibrotic condition,” and “fibrotic disease” which include any disorder, condition or disease characterized by fibrosis. Examples of fibrotic disorders include, but are not limited to vascular fibrosis, pulmonary fibrosis (e.g., idiopathic pulmonary fibrosis), pancreatic fibrosis, liver fibrosis (e.g., following liver transplantation or following hepatitis C virus infection), renal fibrosis (e.g., interstitial fibrosis in focal segmental glomerulosclerosis and nephrogenic systemic fibrosis), musculoskeletal fibrosis, cardiac fibrosis (e.g., endomyocardial fibrosis, idiopathic myocardiopathy), skin fibrosis (e.g., scleroderma, post-traumatic, operative cutaneous scarring, keloids and cutaneous keloid formation), eye fibrosis (e.g., glaucoma, sclerosis of the eyes, conjunctival and corneal scarring, and pterygium), progressive systemic sclerosis (PSS), chronic graft versus-host disease, Peyronie's disease, post-cystoscopic urethral stenosis, idiopathic and pharmacologically induced retroperitoneal fibrosis, mediastinal fibrosis, progressive massive fibrosis, proliferative fibrosis, neoplastic fibrosis, and fibrosis caused by surgical implantation of an artificial organ. Other diseases, disorders, and conditions associated with fibrosis include, for example, cirrhosis which can result from fibrosis of the liver, diffuse parenchymal lung disease, post-vasectomy pain syndrome, tuberculosis which can cause fibrosis of the lungs, sickle-cell anemia may cause enlargement and ultimately fibrosis of the spleen, rheumatoid arthritis, and Crohn's Disease which can cause repeated inflammation and healing of intestinal tissue resulting in fibrosis of the intestinal wall. Fibrosis also occurs as a complication of haemochromatosis, Wilson's disease, alcoholism, schistosomiasis, viral hepatitis, bile duct obstruction, exposure to toxins, and metabolic disorders.

In one embodiment of the invention, the fibrosis is pulmonary fibrosis, e.g., idiopathic pulmonary fibrosis (IPF), also known as cryptogenic fibrosing alveolitis, and IPF/UIP (usual interstitial pneumonia).

Fibrosis may be diagnosed in a subject using methods known to one of ordinary skill in the art. For example, a fibrosis may be diagnosed using routine blood chemistry analysis, ultrasound, radiography, CT, MRI, biopsy and histological examination. Genetic testing (e.g., of the CFTR gene) may also be used to diagnose fibrosis in a subject.

As used herein, the phrase “progression of fibrosis in a subject having fibrosis” refers to the survival rate determined from the beginning of symptoms of fibrosis. A subject may be classified as a “rapid progressor” (or as having “rapid disease progression”) or as a “slow progressor” (or as having “slow disease progression”).

A “rapid progressor” is a subject that survives for less than about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, or less than about 23 months following the onset of symptoms.

A “slow progressor” is a subject that survives for more than about 23 months, about 24 months, about 25 months, about 26 months, about 27 months, about 28 months, about 29 months, about 30 months, about 31 months, about 32 months, about 33 months, about 34 months, about 35 months, about 36 months, about 37 months, about 38 months, about 39 months, about 40 months, about 41 months, about 42 months, about 43 months, about 44 months, about 45 months, about 46 months, about 47 months, about 48 months, about 49 months, about 50 months, about 51 months, about 52 months, about 52 months, about 54 months, about 55 months, about 56 months, about 57 months, about 58 months, about 59 months, about 60 months, about 61 months, about 62 months, about 63 months, about 64 months, about 65 months, about 66 months, about 67 months, about 68 months, about 69 months, about 70 months, about 71 months, about 72 months, about 73 months, about 74 months, about 75 months, about 76 months, about 77 months, about 78 months, about 79 months, about 80 months, about 81 months, about 82 months, about 83 months, about 84 months, about 85 months, about 86 months, about 87 months, about 88 months, about 89 months, about 90 months, about 91 months, about 92 months, about 93 months, about 94 months, about 95 months, about 96 months, about 97 months, about 98 months, about 99 months, about 100 months, about 101 months, about 102 months, about 103 months, about 104 months, about 105 months, about 106 months, about 107 months, about 108 months, about 109 months, about 110 months, about 111 months, about 112 months, about 113 months, about 114 months, about 115 months, about 116 months, about 117 months, about 118 months, about 119 months, about 120 months, or longer following the onset of symptoms.

In addition, a subject with rapid disease progression may have an oxygen saturation level (SpO₂) at rest below the median level of a slow progressor, e.g., at about six months following diagnosis of fibrosis. SpO₂ levels are an indicator of the percentage of hemoglobin saturated with oxygen at the time of the measurement and may be determined by using, for example, pulse oximetry.

A slow progressor and a rapid progressor may have similar physiologic and radiographic features at the time of onset of symptoms and/or presentation to a physician.

Furthermore, among “slow progressors” there is a sub-group of patients which have an acute clinical deterioration (“acute exacerbation of IPF” (“AE-IPF”)) which precedes the terminal phase of the illness. The symptoms of AE-IPF include, for example, a sudden worsening of dyspnea, newly developing diffuse radiographic opacities, worsening hypoxemia, and absence of infectious pneumonia, heart failure, or sepsis. A rapid progressor, as defined herein, is not a subject with AE-IPF.

As used herein, the term “Toll-like receptor” or “TLR” refers to the single membrane-spanning non-catalytic receptors that recognize structurally conserved molecules derived from microbes. TLRs together with the Interleukin-1 receptor, e.g., IL-1 receptor and IL-18 receptors, form a receptor superfamily, known as the “Interleukin-1 Receptor/Toll-Like Receptor Superfamily.” Members of this family are characterized structurally by an extracellular leucine-rich repeat (LRR) domain, a conserved pattern of juxtamembrane cysteine residues, and an intracytoplasmic signaling domain (Toll/IL-1 resistance or Toll-IL-1 receptor (TIR)) domain that forms a platform for downstream signaling by recruiting (via TIR-TIR interactions) TIR domain-containing adapters including MyD88, TIR domain-containing adaptor (TIRAP), and TIR domain-containing adaptor inducing IFNβ (TRIF) (L. A. O'Neill, A. G. Bowie (2007) Nat Rev Immunol 7:353).

The nucleotide and amino acid sequences of TLR9 are known in the art and can be found in, for example, gi:20302169, gi:157057165 (TLR9 human and mouse, respectively).

A “higher level of expression” or an “increase in the level of expression” of TLR9 refers to an expression level in a test sample that is greater than the standard error of the assay employed to assess expression, and is preferably at least twice, and more preferably three, four, five, six, seven, eight, nine, or ten or more times the expression level of TLR9 in a control sample (e.g., a sample from a healthy subject not afflicted with fibrosis and/or a sample from a subject(s) having slow disease progression and/or, the average expression level of TLR9 in several control samples).

A “lower level of expression” or a “decrease in the level of expression” of TLR9 refers to an expression level in a test sample that is less than the standard error of the assay employed to assess expression, and preferably at least twice, and more preferably three, four, five, six, seven, eight, nine, or ten or more times less than the expression level of TLR9 in a control sample (e.g., a sample from a subject with rapid disease progression and/or a sample from the subject prior to administration of a portion of a therapy for fibrosis and/or the average expression level of TLR9 in several control samples).

The term “known standard level” or “control level” refers to an accepted or pre-determined expression level of TLR9 which is used to compare TLR9 expression level in a sample derived from a subject. In one embodiment, the control expression level of TLR9 is based the expression level of TLR9 in a sample(s) from a subject(s) having slow disease progression. In another embodiment, the control expression level of TLR9 is based on the expression level in a sample from a subject or subjects having rapid disease progression. In another embodiment, the control expression level of TLR9 is based on the expression level of TLR9 in a sample(s) from an unaffected, i.e., non-disease, subject(s), i.e., a subject who does not have a fibrosis. In yet another embodiment, the control expression level of TLR9 is based on the expression level of TLR9 in a sample from a subject(s) prior to the administration of a therapy for fibrosis. In another embodiment, the control expression level of TLR9 is based on the expression level of TLR9 in a sample(s) from a subject(s) having fibrosis that is not contacted with a test compound. In another embodiment, the control expression level of TLR9 is based on the expression level of TLR9 in a sample(s) from a subject(s) not having fibrosis that is contacted with a test compound. In one embodiment, the control expression level of TLR9 is based on the expression level of TLR9 in a sample(s) from an animal model of fibrosis, a cell, or a cell line derived from the animal model of fibrosis.

In still other embodiments of the invention, a control level of expression of TLR9 is based on the expression level of TLR9 in a sample(s) from the subject having fibrosis which appears to be non-fibrotic. For example, when laparoscopy or other medical procedure reveals the presence of fibrosis in one portion of an organ, the control level of expression of TLR9 may be determined using the non-affected portion of the organ, and this control level of expression may be compared with the level of expression of TLR9 in an affected portion (i.e., fibrotic portion) of the organ.

Alternatively, and particularly as further information becomes available as a result of routine performance of the methods described herein, population-average values for “control” level of expression of TLR9 may be used. In other embodiments, the “control” level of expression of TLR9 may be determined by determining expression level of TLR9 in a subject sample obtained from a subject before the suspected onset of fibrois in the subject, from archived subject samples, and the like.

As used herein, the terms “patient” or “subject” refer to human and non-human animals, e.g., veterinary patients. The term “non-human animal” includes all vertebrates, e.g., mammals and non-mammals, such as non-human primates, mice, rabbits, sheep, dog, cat, horse, cow, chickens, amphibians, and reptiles. In one embodiment, the subject is a human.

The term “sample” as used herein refers to a collection of similar cells or tissue isolated from a subject, as well as tissues, cells and fluids present within a subject. The term “sample” includes any body fluid (e.g., blood fluids, lymph, gynecological fluids, cystic fluid, urine, ocular fluids and fluids collected by bronchial lavage and/or peritoneal rinsing), or a cell from a subject. In one embodiment, the tissue or cell is removed from the subject. In another embodiment, the tissue or cell is present within the subject. Other subject samples, include tear drops, serum, cerebrospinal fluid, feces, sputum and cell extracts. In one embodiment, the biological sample contains protein molecules from the test subject. In another embodiment, the biological sample may contain mRNA molecules from the test subject or genomic DNA molecules from the test subject.

The progression of fibrosis is “slowed” if at least one symptom of the fibrosis is expected to be or is alleviated, terminated, slowed, delayed, or prevented.

A kit is any manufacture (e.g. a package or container) comprising at least one reagent, e.g. a probe or primer, for specifically detecting TLR9, the manufacture being promoted, distributed, or sold as a unit for performing the methods of the present invention.

### II. Uses of the Invention

A. Prognostic Methods

The present invention provides methods for predicting the progression of fibrosis in a subject having fibrosis. The methods include determining the level of expression of Toll-like receptor 9 (TLR9) in a sample obtained from the subject, and comparing the level of expression of TLR9 in the sample from the subject with the level of expression of TLR9 in a control sample, wherein an increase in the level of expression of TLR9 in the sample from the subject as compared to the level of expression of TLR9 in the control sample is an indication that the fibrosis will rapidly progress.

In one embodiment, determining the level of expression of TLR9 in a sample includes contacting a sample derived from the subject with an agent which transforms the sample in a manner such that the level of expression of TLR9 is detected.

Any sample obtained from a subject having fibrosis may be used to determine the level of expression of TLR9. For example, the sample may be any fluid or sub-component thereof, e.g., fluids collected by bronchial lavage, blood fluids, serum, plasma, vomit, intra-articular fluid, saliva, lymph, cystic fluid, urine, fluids collected by peritoneal rinsing, synovial fluid, or gynecological fluids, obtained from the subject. The sample may also be any tissue or fragment or sub-component thereof, e.g., bronchi, lung, bone, connective tissue, cartilage, liver, kidney, muscle tissue, heart, pancreas, bone and skin, obtained from the subject.

Techniques or methods for obtaining samples from a subject are well known in the art and include, for example, obtaining samples by a swab, a wash, aspiration, or a biopsy. Isolating sub-components of fluid or tissue samples (e.g., cells or RNA or DNA) may be accomplished using well known techniques in the art and those described in the Examples section below.

In one aspect of the invention, the prognostic methods include obtaining a sample from a subject having fibrosis, culturing the sample in duplicate, and determining responsiveness of one of the samples to TGFβ and determining responsiveness of the duplicate sample to CpG, wherein a response of the sample cultured with TGFβ and a response of the duplicate sample cultured with CpG is an indication that the fibrosis will rapidly progress. Such methods may further comprise determining the level of expression of TLR9 or , in certain embodiments, may not comprise determining the level of expression of TLR9.

The methods of the invention may further include determining the presence or absence of unmethylated CpG in the sample from the subject. Determining the presence or absence of unmethylated CpG may include, for example, the use of bisulfite treatment of DNA, methylation-sensitive restriction enzymes, and/or methylation specific PCR (as described in, for example, U.S. Pat. No. 5,786,146, the entire contents of which are incorporated herein by reference).

The methods of the invention may also further include determining the presence or absence of a gammaherpesvirus (e.g., a Lymphocryptovirus, a Rhadinovirus, a Macavirus, and a Percavirus) in the sample from the subject. Determining the presence or absence of a gammaherpesvirus may include, for example, serological analysis, immunoflourescent staining, PCR analysis, and/or culturing of virus from subject samples.

In addition, the methods of the invention may further include determining the level of expression in the sample of a marker selected from the group consisting of annexin 1, alpha smooth muscle actin, neutrophil elastase, KL-6, ST2, IL-8, alpha defensin, beta3-endonexin, serine protease inhibitor, Kazal type, plasminogen activator inhibitor-1, HPS3, Rab38, Smad6, ADAMTS7, CXCR6, Bc12-L-10, and MMP-9. The level of expression of any of these markers may be determined using any of the methods and techniques described herein.

The nucleotide an amino acid sequence of annexin 1 are known and may be found in, for example, GenBank Reference No. GI:4502100; the nucleotide an amino acid sequence of alpha smooth muscle actin are known and may be found in, for example, GenBank Reference No. GI:47078293; the nucleotide an amino acid sequence of neutrophil elastase are known and may be found in, for example, GenBank Reference No. GI:58530849; the nucleotide an amino acid sequence of KL-6 are known and may be found in, for example, GenBank Reference Nos.GI:67189006, GI:67189068, GI:113206023GI:113206025, GI:113206027, GI:113206029, and GI:65301116; the nucleotide an amino acid sequence of ST2 are known and may be found in, for example, GenBank Reference Nos. GI:27894327 and GI:27894323; the nucleotide an amino acid sequence of 1L-8 are known and may be found in, for example, GenBank Reference No. GI:28610153; the nucleotide an amino acid sequence of alpha defensin are known and may be found in, for example, GenBank Reference No. GI:12621915; the nucleotide an amino acid sequence of beta3-endonexin are known and may be found in, for example, GenBank Reference No. GI:27597074; the nucleotide an amino acid sequence of serine protease inhibitor, Kazal type are known and may be found in, for example, GenBank Reference No. GI:195234783; the nucleotide an amino acid sequence of plasminogen activator inhibitor-1 are known and may be found in, for example, GenBank Reference No. GI:169790801; the nucleotide an amino acid sequence of HPS3 are known and may be found in, for example, GenBank Reference No. G1:28416957; the nucleotide an amino acid sequence of Rab38 are known and may be found in, for example, GenBank Reference No. GI:11641236; the nucleotide an amino acid sequence of Smad6 are known and may be found in, for example, GenBank Reference Nos. GI:236465444 and GI:236465646; the nucleotide an amino acid sequence of ADAMTS7 are known and may be found in, for example, GenBank Reference No. GI:133925806; the nucleotide an amino acid sequence of CXCR6 are known and may be found in, for example, GenBank Reference No. GI:5730105; the nucleotide an amino acid sequence of Bcl2-L-10 are known and may be found in, for example, GenBank Reference No. GI:20336328; and the nucleotide an amino acid sequence of MMP-9 are known and may be found in, for example, GenBank Reference No. GI:74272286.

Furthermore, the methods of the present invention can be practiced in conjunction with any other method used by the skilled practitioner to prognose the progression of fibrosis in a subject having fibrosis. For example, the methods of the invention may be performed in conjunction with any clinical measurement of fibrosis known in the art including cytological and/or detection (and quantification, if appropriate) of other molecular markers.

The level of expression of TLR9 in a sample obtained from a subject may be determined by any of a wide variety of well known techniques and methods, which transform TLR9 within the sample into a moiety that can be detected and quantified. Non-limiting examples of such methods include analyzing the sample using immunological methods for detection of proteins, protein purification methods, protein function or activity assays, nucleic acid hybridization methods, nucleic acid reverse transcription methods, and nucleic acid amplification methods, immunoblotting, Western blotting, Northern blotting, electron microscopy, mass spectrometry, e.g., MALD1-TOF and SELDI-TOF, immunoprecipitation, immunofluorescence, immunohistochemistry, enzyme linked immunosorbent assays (ELISAs), e.g., amplified ELISA, quantitative blood based assays, e.g., serum ELISA, quantitative urine based assays, flow cytometry, Southern hybridizations, array analysis, and the like, and combinations or sub-combinations thereof.

For example, an mRNA sample may be obtained from the sample from the subject (e.g., bronchial lavage, mouth swab, biopsy, or peripheral blood mononuclear cells, by standard methods) and expression of mRNA(s) encoding TLR9 in the sample may be detected and/or determined using standard molecular biology techniques, such as PCR analysis. A preferred method of PCR analysis is reverse transcriptase-polymerase chain reaction (RT-PCR). Other suitable systems for mRNA sample analysis include microarray analysis (e.g., using Affymetrix's microarray system or Illumina's BeadArray Technology).

It will be readily understood by the ordinarily skilled artisan that essentially any technical means established in the art for detecting the level of expression of TLR9 at either the nucleic acid or protein level, can be used to determine the level of expression of TLR9 as discussed herein.

In one embodiment, the level of expression of TLR9 in a sample is determined by detecting a transcribed polynucleotide, or portion thereof, e.g., mRNA, or cDNA, of the TLR9 gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, RNase protection assays (Melton et al., Nuc. Acids Res. 12:7035), Northern blotting, in situ hybridization, and microarray analysis.

In one embodiment, the level of expression of TLR9 is determined using a nucleic acid probe. The term “probe”, as used herein, refers to any molecule that is capable of selectively binding to a specific TLR9. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction (PCR) analyses and probe arrays. One method for the determination of mRNA levels involves contacting the isolated mRNA with a nucleic acid molecule (probe) that can hybridize to TLR9 mRNA. The nucleic acid probe can be, for example, a full-length cDNA, or a portion thereof, such as an oligonucleotide of at least about 7, 10, 15, 20, 25, 30, 35, 40, 45, 50, 100, 250 or about 500 nucleotides in length and sufficient to specifically hybridize under stringent conditions to TLR9 genomic DNA.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in determining the level of TLR9 mRNA.

An alternative method for determining the level of expression of TLR9 in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et at (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of TLR9 is determined by quantitative fluorogenic RT-PCR (i.e., the TaqMan™ System). Such methods typically utilize pairs of oligonucleotide primers that are specific for TLR9. Methods for designing oligonucleotide primers specific for a known sequence are well known in the art.

The expression levels of TLR9 mRNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of TLR9 expression level may also comprise using nucleic acid probes in solution.

In one embodiment of the invention, microarrays are used to detect the level of expression of TLR9. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

In certain situations it may be possible to assay for the expression of TLR9 at the protein level, using a detection reagent that detects the protein product encoded by the mRNA of TLR9. For example, if an antibody reagent is available that binds specifically to TLR9 protein product to be detected, and not to other proteins, then such an antibody reagent can be used to detect the expression of TLR9 in a cellular sample from the subject, or a preparation derived from the cellular sample, using standard antibody-based techniques known in the art, such as FACS analysis, and the like.

Other known methods for detecting TLR9 at the protein level include methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, and Western blotting.

Proteins from samples can be isolated using techniques that are well known to those of skill in the art. The protein isolation methods employed can, for example, be those described in Harlow and Lane (Harlow and Lane, 1988, Antibodies: A Laboratory Manual, Cold Spring Harbor Laboratory Press, Cold Spring Harbor, N.Y.).

In one embodiment, antibodies, or antibody fragments, are used in methods such as Western blots or immunofluorescence techniques to detect the expressed proteins. Antibodies for determining the expression of TLR9 are commercially available from, for example, Imgenex (San Diego, Calif.; www.imgenex.com/Toll-likeReceptors.php), e.g., the TLR9 specific antibodies IMG-431 and IMG-305A; lnvivogen (San Diego, Calif.; www.invivogen.com/family.php?ID=162&IDcat=2&ID_sscat=102), e.g., the TLR9 specific antibodies mab-mtlr9; Santa Cruz Biotechnology, Inc. (Santa Cruz, Calif.; www.scbt.com/table-tlr.html), e.g., the TLR9 specific antibodies sc-52966, sc-13218, and sc-25468; and Cambridge Bioscience (Cambridge, UK; www.bioscience.co.uk/newsDetail.php?newsID=107368), e.g., the TLR9 specific antibodies HM1042, 905-730-100, IMG-305A, and IMG-431.

It is generally preferable to immobilize either the antibody or proteins on a solid support for Western blots and immunofluorescence techniques. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

Other standard methods include immunoassay techniques which are well known to one of ordinary skill in the art and may be found in Principles And Practice Of Immunoassay, 2nd Edition, Price and Newman, eds., MacMillan (1997) and Antibodies, A Laboratory Manual, Harlow and Lane, eds., Cold Spring Harbor Laboratory, Ch. 9 (1988), each of which is incorporated herein by reference in its entirety.

Antibodies used in immunoassays to determine the level of expression of TLR9, may be labeled with a detectable label. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. In one embodiment, the antibody is labeled, e.g. a radio-labeled, chromophore-labeled, fluorophore-labeled, or enzyme-labeled antibody. In another embodiment, an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with TLR9.

In one embodiment of the invention, proteomic methods, e.g., mass spectrometry, are used. Mass spectrometry is an analytical technique that consists of ionizing chemical compounds to generate charged molecules (or fragments therof) and measuring their mass-to-charge ratios. In a typical mass spectrometry procedure, a sample is obtained from a subject, loaded onto the mass spectrometry, and its components (e.g., TLR9) are ionized by different methods (e.g., by impacting them with an electron beam), resulting in the formation of charged particles (ions). The mass-to-charge ratio of the particles is then calculated from the motion of the ions as they transit through electromagnetic fields.

For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis Markers 19:229; Petricoin, E. F., et al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to determine the expression level of TLR9.

Furthermore, in vivo techniques for determination of the expression level of TLR9 include introducing into a subject a labeled antibody directed against TLR9, which binds to and transforms TLR9 into a detectable molecule. As discussed above, the presence, level, or even location of the detectable TLR9 in a subject may be detected determined by standard imaging techniques.

In general, it is preferable that the difference between the level of expression of TLR9 in a sample from a subject having fibrosis and the amount of TLR9 in a control sample, is as great as possible. Although this difference can be as small as the limit of detection of the method for determining the level of expression it is preferred that the difference be at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater than the standard error of the assessment method.

B. Identification of Compounds That Can Slow Down the Progression of Fibrosis in A Subject Having Fibrosis

Using the methods described herein, a variety of molecules, particularly molecules sufficiently small to be able to cross the cell membrane, may be screened in order to identify molecules which modulate, e.g., decrease, the expression and/or activity of TLR9. Compounds so identified can be provided to a subject having fibrosis in order to inhibit or slow down the progression of fibrosis in the subject.

Methods for identifying a compound that can slow down the progression of fibrosis in a subject having fibrosis (also referred to herein as screening assays) include separately contacting an aliquot of a sample from the subject with each member of a library of compounds; determining the effect of a member of the library of compounds on the level of expression of Toll-like receptor 9 (TLR9) (or the activity of TLR9) in each of the aliquots; and selecting a member of the library of compounds which decreases the level of expression and/or the activity of TLR9 in an aliquot as compared to the level of expression of TLR9 in a control sample, thereby identifying a compound that can slow down the progression of fibrosis in a subject having fibrosis.

As used interchangeably herein, the terms “TLR9 activity” and “biological activity of TLR9” include activities exerted by TLR9 protein on TLR9 responsive cell or tissue, e.g., a dendritic cell (DC), or on TLR9 nucleic acid molecule or protein target molecule, as determined in vivo, and/or in vitro, according to standard techniques. A TLR9 activity can be a direct activity, such as an association with a TLR9-target molecule e.g., association or interaction with an adaptor molecule, e.g., MyD88. Alternatively, TLR9 activity is an indirect activity, such as a downstream biological event mediated by interaction of the TLR9 protein with a TLR9-target molecule, e.g., EDEM or other molecule in a signal-transduction pathway involving TLR9. The biological activities of TLR9 are known in the art and include e.g., lymphocyte proliferation, cytokine production, activation of nuclear factor κB (NF-κB), response to CpG DNA, maturation of DCs, and/or a T-helper type-1 response.

Methods for determining the effect of a compound on the expression and/or activity of TLR9 are known in the art and/or described herein.

A variety of test compounds can be evaluated using the screening assays described herein. The term “test compound” includes any reagent or test agent which is employed in the assays of the invention and assayed for its ability to influence the expression and/or activity of TLR9. More than one compound, e.g., a plurality of compounds, can be tested at the same time for their ability to modulate the expression and/or activity of TLR9 in a screening assay. The term “screening assay” preferably refers to assays which test the ability of a plurality of compounds to influence the readout of choice rather than to tests which test the ability of one compound to influence a readout. Preferably, the subject assays identify compounds not previously known to have the effect that is being screened for. In one embodiment, high throughput screening can be used to assay for the activity of a compound.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), 6) mutant forms of TLR9 molecules, e.g., dominant negative mutant forms of the molecules, 7) nucleic acids, 8) carbohydrates, and 9) natural product extract compounds.

Test compounds can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP. '409), plasmids (Cull et al. (1992) Proc Natl Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222 :301-310; Ladner supra.).

Compounds identified in the screening assays can be used in methods of modulating one or more of the biological responses regulated by TLR9, e.g., fibrosis. It will be understood that it may be desirable to formulate such compound(s) as pharmaceutical compositions (described supra) prior to contacting them with cells.

Once a test compound is identified by one of the variety of methods described hereinbefore, the selected test compound (or “compound of interest”) can then be further evaluated for its effect on cells, for example by contacting the compound of interest with cells either in vivo (e.g., by administering the compound of interest to a subject or animal model) or ex vivo (e.g., by isolating cells from the subject or animal model and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

Computer-based analysis of TLR9 with a known structure can also be used to identify molecules which will bind to TLR9. Such methods rank molecules based on their shape complementary to a receptor site. For example, using a 3-D database, a program such as DOCK can be used to identify molecules which will bind to TLR9. See DesJarlias et al. (1988) J. Med. Chem. 31:722; Meng et al. (1992) J. Computer Chem. 13:505; Meng et al. (1993) Proteins 17:266; Shoichet et al. (1993) Science 259:1445. In addition, the electronic complementarity of a molecule to TLR9 can be analyzed to identify molecules which bind to TLR9. This can be determined using, for example, a molecular mechanics force field as described in Meng et al. (1992) J. Computer Chem. 13:505 and Meng et al. (1993) Proteins 17:266. Other programs which can be used include CLIX which uses a GRID force field in docking of putative ligands. See Lawrence et al. (1992) Proteins 12:31; Goodford et al. (1985) J. Med. Chem. 28:849; Boobbyer et al. (1989) J. Med. Chem. 32:1083.

The instant invention also pertains to compounds identified using the foregoing screening assays.

C. Methods for Monitoring the Effectiveness of a Therapy in Reducing the Progression of Fibrosis in a Subject Having Fibrosis.

Methods for monitoring the effectiveness of a therapy or treatment regimen (e.g., removal of the underlying cause (e.g., toxin or infectious agent), suppression of inflammation (using, e.g., corticosteroids, IL-I receptor antagonists, or other agents), gamma interferon or antioxidant treatment), promotion of matrix degradation, or any other therapeutic approach useful for reducing or slowing the progression of fibrosis and/or treating fibrosis in a subject having fibrosis), are also provided. In these methods the level of expression of TLR9 in a pair of samples (a first sample not subjected to the treatment regimen and a second sample subjected to at least a portion of the treatment regimen) is assessed. A decrease in the level of expression of TLR9 in the first sample, relative to the second sample, is an indication that the therapy is effective in reducing the progression of fibrosis in the subject having fibrosis.

In one embodiment, the therapy comprises use of an anti-CCL21 antibody (Pirece et al AJP 2007 and Pierce et al ERJ 2007) an anti-PDGFβ antibody, an anti-IL-13 antibody, an anti-TGFβ antibody, an anti-integrin antibody, a kinase inhibitor, an LBA receptor inhibitor, or a BMP modulator. In another embodiment, the therapy comprises TLR9 inhibitor, such as an immunoregulatory sequences (IRS) (see, e.g., U.S. Pat. No. 6,225,292) and other DNA sequences (see, e.g., Stunz L L. et al. (2002) Eur J Immunol. 32(5): 1212-22).

D. Methods for Selecting a Subject for Participation in a Clinical Trial for a Treatment of Fibrosis

Noble, P., et al. have recently reported that the variability of fibrosis progression has confounded the data obtained in clinical trials of fibrosis treatments (see, e.g., Noble, P., et al. (2009) Am. J. Respir Crit Care Med. 179:A1129). The discovery by the present inventors that the level of expression of TLR9 distinguishes rapidly progressing patients from slowly progressing patients serves to reduce the variability in a patient population participating in a clinical trial of treatments of fibrosis. Determining the level of expression of TLR9 is also useful for selecting a subject for participation in a clinical trial for a treatment of fibrosis by identifying, for example, a subject most likely to benefit from a new treatment or from a known treatment, e.g., a known treatment with a high risk profile of adverse side effects. For example, physicians typically select therapeutic regimens for subject treatment based upon the expected net benefit to the subject. The net benefit is derived from the risk to benefit ratio. The present methods permit selection of subjects who are more likely to benefit by intervention, thereby aiding the physician in selecting a therapeutic regimen. This might include using drugs with a higher risk profile where the likelihood of expected benefit has increased. Likewise, clinical investigators may desire to select for clinical trials a population with a high or low likelihood of obtaining a net benefit with a particular protocol. The methods described herein can be used by clinical investigators to select such a subject. Thus, in some embodiments, the methods provide entry criteria and methods for selecting a subject for clinical trials, by selecting subjects that are rapid progressors and/or slow progressors.

Methods for selecting a subject for participation in a clinical trail include determining the level of expression of Toll-like receptor 9 (TLR9) in a sample from a subject having fibrosis, and comparing the level of expression of TLR9 in the sample from the subject to the level of expression of TLR9 in a control sample, wherein a higher level of expression of TLR9 in the sample from the subject as compared to the level of expression of TLR9 in the control sample is an indication that the subject should participate in the clinical trial, thereby selecting a subject for participation in a clinical trial for a treatment of fibrosis. In another embodiment, a lower level of expression of TLR9 in the sample from the subject as compared to the level of expression of TLR9 in the control sample is an indication that the subject should participate in the clinical trial.

E. Methods for Inhibiting the Progression of Fibrosis Using TLR9 Antagonists

The present invention also provides methods for inhibiting the progression of fibrosis in a cell, such as a pulmonary cell, a liver cell, a kidney cell, a cardiac cell, a musculoskeletal cell, a skin cell, an eye cell, or a pancreatic cell. The methods include contacting the cell with an effective amount of TLR9 antagonist, thereby inhibiting progression of fibrosis in the cell.

The present invention further provides methods for inhibiting progression of fibrosis in a subject. The methods include administering an effective amount of TLR9 antagonist to the subject, thereby inhibiting progression of fibrosis in the subject.

The methods of “inhibiting progression of fibrosis” include administration of TLR9 antagonist to a subject in order to cure or to prolong the health or survival of a subject beyond that expected in the absence of such treatment. In one embodiment, “inhibiting the progression of fibrosis” includes reducing the severity of, or amelioration of one or more symptoms of a fibrotic disease or condition. For example, “inhibiting the progression of fibrosis” includes the alleviation of a fibrotic disease symptom (e.g., shortness of breath, fatigue, cough, weight loss, loss of appetite associated with pulmonary fibrosis or anorexia, fatigue, or weight loss), in a subject by at least 5%, 6%, 7%, 8%, 9%, 10%, 11%, 12%, 13%, 14%, 15%, 16%, 17%, 18%, 19%, 20%, 25%, 30%,

The terms “patient” or “subject” as used herein is intended to include human and veterinary patients. In a particular embodiment, the subject is a human. The term “non-human animal” includes all vertebrates, e.g., mammals and non-mammals, such as non-human primates, mice, rabbits, sheep, dog, cow, chickens, amphibians, and reptiles.

As used herein, the term “antagonist” refers to any moiety which downmodulates TLR9 activity, including moieties which downregulate TLR9 expression or inhibit TLR9 function. In one aspect of the invention, the antagonist may be any moiety which directly antagonizes TLR9. For example, in one embodiment, the antagonist is a peptide or antibody which binds to TLR9 and prevents TLR9 from binding to its ligand (e.g., CpG), thereby inhibiting TLR9 signaling. In another embodiment, the antagonist is a peptide or antibody which binds to the ligand of TLR9 and prevents TLR9 from binding to this ligand. In another aspect of the invention, the moiety indirectly antagonizes TLR9 by modulating the activity of downstream mediators in a TLR9 signaling pathway.

Representative antagonists, include, but are not limited to, antibodies, nucleic acids (e.g., antisense molecules, such as ribozymes and RNA interfering agents), immunoconjugates (e.g., an antibody conjugated to a therapeutic agent), small molecule inhibitors, fusion proteins, adnectins, aptamers, anticalins, lipocalins, and TLR9-derived peptidic compounds.

In one embodiment of the invention, the therapeutic and diagnostic methods described herein employ an antibody that binds, e.g., directly to or indirectly to, and inhibits TLR9 activity and/or down-modulates TLR9 expression.

The term “antibody” or “immunoglobulin,” as used interchangeably herein, includes whole antibodies and any antigen binding fragment (i.e., “antigen-binding portion”) or single chains thereof. An “antibody” comprises at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., TLR9). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single aim of an antibody, (v) a dAb including VH and VL domains; (vi) a dAb fragment (Ward et al. (1989) Nature 341, 544-546), which consists of a V_{H} domain; (vii) a dAb which consists of a VH or a VL domain; and (viii) an isolated complementarity determining region (CDR) or (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242, 423-426; and Huston et al. (1988) Proc. Natl. Acad. Sci. USA 85, 5879-5883). Such single chain antibodies are also intended to be encompassed within the term “antigen-binding portion” of an antibody. These antibody fragments are obtained using conventional techniques known to those with skill in the art, and the fragments are screened for utility in the same mariner as are intact antibodies. Antigen-binding portions can be produced by recombinant DNA techniques, or by enzymatic or chemical cleavage of intact immunoglobulins.

The term “antibody”, as used herein, includes polyclonal antibodies, monoclonal antibodies, chimeric antibodies, humanized antibodies, and human antibodies, and those that occur naturally or are recombinantly produced according to methods well known in the art.

In one embodiment, an antibody for use in the methods of the invention is a bispecific antibody. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79, 315-321; Kostelny et al. (1992) J. Immunol. 148, 1547-1553.

In another embodiment, an antibody for use in the methods of the invention is a camelid antibody as described in, for example, PCT Publication WO 94/04678, the entire contents of which are incorporated herein by reference.

A region of the camelid antibody that is the small, single variable domain identified as V_{HH} can be obtained by genetic engineering to yield a small protein having high affinity for a target, resulting in a low molecular weight, antibody-derived protein known as a “camelid nanobody”. See U.S. Pat. No. 5,759,808; see also Stijlemans et al., 2004 J. Biol. Chem. 279: 1256-1261; Dumoulin et al., 2003 Nature 424: 783-788; Pleschberger et al., 2003 Bioconjugate Chem. 14: 440-448; Cortez-Retamozo et al., 2002 Int. J. Cancer 89: 456-62; and Lauwereys. et al., 1998 EMBO J. 17: 3512-3520. Engineered libraries of camelid antibodies and antibody fragments are commercially available, for example, from Ablynx, Ghent, Belgium. Accordingly, a feature of the present invention is a camelid nanobody having high affinity for TLR9.

In other embodiments of the invention, an antibody for use in the methods of the invention is a diabody, a single chain diabody, or a di-diabody.

Diabodies are bivalent, bispecific molecules in which V_{H} and V_{L} domains are expressed on a single polypeptide chain, connected by a linker that is too short to allow for pairing between the two domains on the same chain. The V_{H} and V_{L} domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure V_{HA}-V_{LB} and V_{HB}-V_{LA} (V_{H}-V_{L} configuration), or V_{LA}-V_{HB} and V_{LB}-V_{HA} (V_{L}-V_{H} configuration) within the same cell. Most of them can be expressed in soluble form in bacteria.

Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack, 1997 Immunotechnology, 3(2): 83-105; Ridgway et al., 1996 Protein Eng., 9(7):617-21).

A diabody can be fused to Fc to generate a “di-diabody” (see Lu et al., 2004 J. Biol. Chem., 279(4):2856-65).

TLR9 binding molecules that exhibit functional properties of antibodies but derive their framework and antigen binding portions from other polypeptides (e.g., polypeptides other than those encoded by antibody genes or generated by the recombination of antibody genes in vivo) may also be used in the methods of the present invention. The antigen binding domains (e.g., TLR9 binding domains) of these binding molecules are generated through a directed evolution process. See U.S. Pat. No. 7,115,396. Molecules that have an overall fold similar to that of a variable domain of an antibody (an “immunoglobulin-like” fold) are appropriate scaffold proteins. Scaffold proteins suitable for deriving antigen binding molecules include fibronectin or a fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

To generate non-antibody binding molecules, a library of clones is created in which sequences in regions of the scaffold protein that form antigen binding surfaces (e.g., regions analogous in position and structure to CDRs of an antibody variable domain immunoglobulin fold) are randomized. Library clones are tested for specific binding to the antigen of interest (e.g., TLR9) and for other functions (e.g., inhibition of biological activity of TLR9). Selected clones can be used as the basis for further randomization and selection to produce derivatives of higher affinity for the antigen.

High affinity binding molecules are generated, for example, using the tenth module of fibronectin III (¹⁰Fn3) as the scaffold, described in U.S. Pat. Nos. 6,818,418 and 7,115,396; Roberts and Szostak, 1997 Proc. Natl. Acad. Sci USA 94:12297; U.S. Pat. No. 6,261,804; U.S. Pat. No. 6,258,558; and Szostak et al. WO98/31700, the entire contents of each of which are incorporated herein by reference.

Non-antibody binding molecules can be produced as dimers or multimers to increase avidity for the target antigen. For example, the antigen binding domain is expressed as a fusion with a constant region (Fc) of an antibody that forms Fc-Fc dimers. See, e.g., U.S. Pat. No. 7,115,396, the entire contents of which are incorporated herein by reference.

The therapeutic methods of the invention also may be practiced through the use of antibody fragments and antibody mimetics. As detailed below, a wide variety of antibody fragment and antibody mimetic technologies have now been developed and are widely known in the art. While a number of these technologies, such as domain antibodies, Nanobodies, and UniBodies make use of fragments of, or other modifications to, traditional antibody structures, there are also alternative technologies, such as Adnectins, Affibodies, DARPins, Anticalins, Avimers, and Versabodies that employ binding structures that, while they mimic traditional antibody binding, are generated from and function via distinct mechanisms. Some of these alternative structures are reviewed in Gill and Damle (2006) 17: 653-658.

Domain Antibodies (dAbs) are the smallest functional binding units of antibodies, corresponding to the variable regions of either the heavy (VH) or light (VL) chains of human antibodies. Domantis has developed a series of large and highly functional libraries of fully human VH and VL dAbs (more than ten billion different sequences in each library), and uses these libraries to select dAbs that are specific to therapeutic targets. In contrast to many conventional antibodies, domain antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; U.S. Ser. No. 2004/0110941; European patent application No. 1433846 and European Patents 0368684 & 0616640; WO05/035572, WO04/101790, WO04/081026, WO04/058821, WO04/003019 and WO03/002609, the contents of each of which is herein incorporated by reference in its entirety.

Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a perfectly stable polypeptide harboring the full antigen-binding capacity of the original heavy-chain antibody. Nanobodies have a high homology with the VH domains of human antibodies and can be further humanized without any loss of activity.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see, e.g., WO 06/079372, which is herein incorporated by reference in its entirety) is a proprietary method for generating Nanobodies against a desired target, based on automated high-throughout selection of B-cells and could be used in the context of the instant invention.

UniBodies are another antibody fragment technology, however this one is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. Further details of UniBodies may be obtained by reference to patent application WO2007/059782, which is herein incorporated by reference in its entirety.

Adnectin molecules are engineered binding proteins derived from one or more domains of the fibronectin protein. In one embodiment, adnectin molecules are derived from the fibronectin type III domain by altering the native protein which is composed of multiple beta strands distributed between two beta sheets. Depending on the originating tissue, fibronectin may contain multiple type III domains which may be denoted, e.g., ¹Fn3, ²Fn3, ³Fn3, etc. Adnectin molecules may also be derived from polymers of ¹⁰Fn3 related molecules rather than a simple monomeric ¹⁰Fn3 structure.

Although the native ¹⁰Fn3 domain typically binds to integrin, ¹⁰Fn3 proteins adapted to become adnectin molecules are altered so to bind antigens of interest, e.g., TLR9. In one embodiment, the alteration to the ¹⁰Fn3 molecule comprises at least one mutation to a beta strand. In a preferred embodiment, the loop regions which connect the beta strands of the ¹⁰Fn3 molecule are altered to bind to an antigen of interest, e.g., TLR9.

The alterations in the ¹⁰Fn3 may be made by any method known in the art including, but not limited to, error prone PCR, site-directed mutagenesis, DNA shuffling, or other types of recombinational mutagenesis which have been referenced herein. In one example, variants of the DNA encoding the ¹⁰Fn3 sequence may be directly synthesized in vitro, and later transcribed and translated in vitro or in vivo. Alternatively, a natural ¹⁰Fn3 sequence may be isolated or cloned from the genome using standard methods (as performed, e.g., in U.S. Pat. Application No. 20070082365), and then mutated using mutagenesis methods known in the art.

An aptamer is another type of antibody-mimetic which may be used in the methods of the present invention. Aptamers are typically small nucleotide polymers that bind to specific molecular targets. Aptamers may be single or double stranded nucleic acid molecules (DNA or RNA), although DNA based aptamers are most commonly double stranded. There is no defined length for an aptamer nucleic acid; however, aptamer molecules are most commonly between 15 and 40 nucleotides long.

Aptamers may be generated using a variety of techniques, but were originally developed using in vitro selection (Ellington and Szostak. (1990) Nature. 346(6287):818-22) and the SELEX method (systematic evolution of ligands by exponential enrichment) (Schneider et al. 1992. J Mol Biol. 228(3):862-9) the contents of which are incorporated herein by reference. Other methods to make and uses of aptamers have been published including Klussmann. The Aptamer Handbook: Functional Oligonucleotides and Their Applications. ISBN: 978-3-527-31059-3; Ulrich et al. 2006. Comb Chem High Throughput Screen 9(8):619-32; Cerchia and de Franciscis. 2007. Methods Mal Biol. 361:187-200; Ireson and Kelland. 2006. Mol Cancer Ther. 2006 5(12):2957-62; U.S. Pat. Nos.: 5,582,981; 5,840,867; 5,756,291; 6,261,783; 6,458,559; 5,792,613; 6,111,095; and US Pat. App. Nos.: 11/482,671; 11/102,428; 11/291,610; and 10/627,543 which are all incorporated herein by reference.

Aptamer molecules made from peptides instead of nucleotides may also be used in the methods of the invention. Peptide aptamers share many properties with nucleotide aptamers (e.g., small size and ability to bind target molecules with high affinity) and they may be generated by selection methods that have similar principles to those used to generate nucleotide aptamers, for example Baines and Colas. 2006. Drug Discov Today. 11(7-8):334-41; and Bickle et al. 2006. Nat Protoc. 1(3):1066-91 which are incorporated herein by reference.

Affibody molecules represent a class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an a-helical bacterial receptor domain, Nat Biotechnol 1997;15:772-7. Ronmark J, Gronlund H, Uhlen M, Nygren P A, Human immunoglobulin A (IgA)-specific ligands from combinatorial engineering of protein A, Eur J Biochem 2002;269:2647-55). Further details of Affibodies and methods of production thereof may be obtained by reference to U.S. Pat. No. 5,831,012 which is herein incorporated by reference in its entirety.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains.

Additional information regarding DARPins and other DRP technologies can be found in U.S. Patent Application Publication No. 2004/0132028 and International Patent Application Publication No. WO 02/20565, both of which are hereby incorporated by reference in their entirety.

Anticalins are an additional antibody mimetic technology, however in this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

Lipocalins are cloned and their loops are subjected to engineering in order to create Anticalins. Libraries of structurally diverse Anticalins have been generated and Anticalin display allows the selection and screening of binding function, followed by the expression and production of soluble protein for further analysis in prokaryotic or eukaryotic systems. Studies have successfully demonstrated that Anticalins can be developed that are specific for virtually any human target protein can be isolated and binding affinities in the nanomolar or higher range can be obtained.

Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains.

Additional information regarding Anticalins can be found in U.S. Pat. No. 7,250,297 and International Patent Application Publication No. WO 99/16873, both of which are hereby incorporated by reference in their entirety.

Another antibody mimetic technology useful in the context of the instant invention are Avimers. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets.

Additional information regarding Avimers can be found in U.S. Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabodies are another antibody mimetic technology that could be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

Additional information regarding Versabodies can be found in U.S. Patent Application Publication No. 2007/0191272 which is hereby incorporated by reference in its entirety.

SMIPs™ (Small Modular ImmunoPharmaceuticals-Trubion Pharmaceuticals) engineered to maintain and optimize target binding, effector functions, in vivo half life, and expression levels. SMIPS consist of three distinct modular domains. First they contain a binding domain which may consist of any protein which confers specificity (e.g., cell surface receptors, single chain antibodies, soluble proteins, etc). Secondly, they contain a hinge domain which serves as a flexible linker between the binding domain and the effector domain, and also helps control multimerization of the SMIP drug. Finally, SM1PS contain an effector domain which may be derived from a variety of molecules including Fc domains or other specially designed proteins. The modularity of the design, which allows the simple construction of SMIPs with a variety of different binding, hinge, and effector domains, provides for rapid and customizable drug design.

More information on SMIPs, including examples of how to design them, may be found in Zhao et al. (2007) Blood 110:2569-77 and the following U.S. Pat. App. Nos. 20050238646; 20050202534; 20050202028; 20050202023; 20050202012; 20050186216; 20050180970; and 20050175614.

In another aspect, the methods of the present invention employ immunoconjugate agents that target TLR9 and which inhibit or down-modulate TLR9. Agents that can be targeted to TLR9 include, but are not limited to, cytotoxic agents, anti-inflammatory agents, e.g., a steroidal or nonsteroidal inflammatory agent, or a cytotoxin antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The term “cytotoxin” or “cytotoxic agent” includes any agent that is detrimental (e.g., kills) to fibrotic tissue. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

lmmunoconjugates can be formed by conjugating (e.g., chemically linking or recombinantly expressing) antibodies to suitable therapeutic agents. Suitable agents include, for example, a cytotoxic agent, a toxin (e.g. an enzymatically active toxin of bacterial, fungal, plant or animal origin, or fragments thereof), and/or a radioactive isotope (i.e., a radioconjugate). Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. A variety of radionuclides are available for the production of radioconjugated antibodies. Examples include ²¹²Bi, ¹³¹I, ¹³¹In, ⁹⁰Y and ¹⁸⁶Re.

Immunoconjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody (see, e.g., WO94/l 1026).

In another embodiment, TLR9 antagonists employed in the methods of the invention are small molecules. As used herein, the term “small molecule” is a term of the art and includes molecules that are less than about 7500, less than about 5000, less than about 1000 molecular weight or less than about 500 molecular weight, and inhibit TLR9 activity. Exemplary small molecules include, but are not limited to, small organic molecules (e.g., Cane et al. 1998. Science 282:63), and natural product extract libraries. In another embodiment, the compounds are small, organic non-peptidic compounds. Like antibodies, these small molecule inhibitors indirectly or directly inhibit the activity of TLR9.

In another embodiment, the TLR9 antagonist employed in the methods of the present invention is an antisense nucleic acid molecule that is complementary to a gene encoding TLR9 or to a portion of that gene, or a recombinant expression vector encoding the antisense nucleic acid molecule. As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

The use of antisense nucleic acids to down-modulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Antisense nucleic acids can be designed according to the rules of Watson and Crick base pairing. The antisense nucleic acid molecule can be complementary to the entire coding region of TLR9 mRNA, but more preferably is an oligonucleotide which is antisense to only a portion of the coding or noncoding region of TLR9 mRNA. For example, the antisense oligonucleotide can be complementary to the region surrounding the translation start site of TLR9 mRNA. An antisense oligonucleotide can be, for example, about 5, 10, 15, 20, 25, 30, 35, 40, 45 or 50 nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules that can be utilized in the methods of the present invention are typically administered to a subject or generated in situ such that they hybridize with or bind to cellular mRNA and/or genomic DNA encoding TLR9 to thereby inhibit expression by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using vectors well known in the art and described in, for example, US20070111230 the entire contents of which are incorporated herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule employed by the methods of the present invention can include an α-anomeric nucleic acid molecule. An a-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 1′-o-methylribonucleotide (Inoue et al. (1987) Nucleic Acids Res. 15:6131-6148) or a chimeric RNA-DNA analogue (Inoue et al. (1987) FEBS Lett. 215:327-330).

In another embodiment, an antisense nucleic acid used in the methods of the present invention is a compound that mediates RNAi. RNA interfering agents include, but are not limited to, nucleic acid molecules including RNA molecules which are homologous to TLR9 or a fragment thereof, “short interfering RNA” (siRNA), “short hairpin” or “small hairpin RNA” (shRNA), and small molecules which interfere with or inhibit expression of a target gene by RNA interference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g., New England Biolabs and Ambion. In one embodiment one or more of the chemistries described above for use in antisense RNA can be employed.

In still another embodiment, an antisense nucleic acid is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, 1988, Nature 334:585-591) can be used to catalytically cleave TLR9 mRNA transcripts to thereby inhibit translation of TLR9 mRNA.

Alternatively, gene expression can be inhibited by targeting nucleotide sequences complementary to the regulatory region of TLR9 (e.g., the promoter and/or enhancers) to form triple helical structures that prevent transcription of the TLR9 gene. See generally, Helene, C., 1991, Anticancer Drug Des. 6(6):569-84; Helene, C. et al., 1992, Ann. N.Y. Acad. Sci. 660:27-36; and Maher, L. J., 1992, Bioassays 14(12):807-15.

In another embodiment, the TLR9 antagonist used in the methods of the present invention is a fusion protein or peptidic compound derived from the TLR9 amino acid sequence. In particular, the inhibitory compound comprises a fusion protein or a portion of TLR9 (or a mimetic thereof) that mediates interaction of TLR9 with a target molecule (e.g., CpG) such that contact of TLR9 with this fusion protein or peptidic compound competitively inhibits the interaction of TLR9 with the target molecule. Such fusion proteins and peptidic compounds can be made using standard techniques known in the art. For example, peptidic compounds can be made by chemical synthesis using standard peptide synthesis techniques and then introduced into cells by a variety of means known in the art for introducing peptides into cells (e.g., liposome and the like).

The in vivo half-life of the fusion protein or peptidic compounds of the invention can be improved by making peptide modifications, such as the addition of N-linked glycosylation sites into TLR9 or conjugating TLR9 to poly(ethylene glycol) (PEG; pegylation), e.g., via lysine-monopegylation. Such techniques have proven to be beneficial in prolonging the half-life of therapeutic protein drugs. It is expected that pegylation of TLR9 polypeptides of the invention may result in similar pharmaceutical advantages.

In addition, pegylation can be achieved in any part of a polypeptide of the invention by the introduction of a nonnatural amino acid. Certain nonnatural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the nonnatural amino acid of choice. Particular nonnatural amino acids that are beneficial for purpose of conjugating moieties to the polypeptides of the invention include those with acetylene and azido side chains. TLR9 polypeptides containing these novel amino acids can then be pegylated at these chosen sites in the protein.

The methods of the invention also contemplate the use of TLR9 antagonists in combination with other therapies. Thus, in addition to the use of TLR9 antagonists, the methods of the invention may also include administering to the subject one or more “standard” therapies for treating fibrotic disorders. For example, the antagonists can be administered in combination with (i.e., together with or linked to (i.e., an immunoconjugate)) cytotoxins, immunosuppressive agents, radiotoxic agents, and/or therapeutic antibodies. Particular co-therapeutics contemplated by the present invention include, but are not limited to, steroids (e.g., corticosteroids, such as Prednisone), immune-suppressing and/or anti-inflammatory agents (e.g., gamma-interferon, cyclophosphamide, azathioprine, methotrexate, penicillamine, cyclosporine, colchicines, antithymocyte globulin, mycophenolate mofetil, and hydroxychloroquine), cytotoxic drugs, calcium channel blockers (e.g., nifedipine), angiotensin converting enzyme inhibitors (ACE) inhibitors, para-aminobenzoic acid (PABA), dimethyl sulfoxide, transforming growth factor-beta (TGF-β) inhibitors, interleukin-5 (IL-5) inhibitors, and pan caspase inhibitors. Additional anti-fibrotic agents that may be used in combination with TLR9 antagonists include lectins (as described in, for example, U.S. Pat. No.: 7,026,283, the entire contents of which are incorporated herein by reference). Pirfenidone (5-methyl-1-phenyl-2-(1H)-pyridone) may also be used in combination with TLR9 antagonists (U.S. Pat. Nos. 3,974,281; 3,839,346; 4,042,699; 4,052,509; 5,310,562; 5,518,729; 5,716,632; and 6,090,822 (the entire contents of all of which are expressly incorporated herein by reference) describe methods for the synthesis and formulation of pirfenidone and specific pirfenidone analogs in pharmaceutical compositions suitable for use in the methods of the present invention).

TLR9 antagonist and the co-therapeutic agent or co-therapy can be administered in the same formulation or separately. In the case of separate administration, the TLR9 antagonist can be administered before, after or concurrently with the co-therapeutic or co-therapy. One agent may precede or follow administration of the other agent by intervals ranging from minutes to weeks. In embodiments where two or more different kinds of therapeutic agents are applied separately to a subject, one would generally ensure that a significant period of time did not expire between the time of each delivery, such that these different kinds of agents would still be able to exert an advantageously combined effect on the target tissues or cells.

In one embodiment, the TLR9 antagonist (e.g., an anti-TLR9 antibody) may be linked to a second binding molecule, such as an antibody (i.e., thereby forming a bispecific molecule) or other binding agent that, for example, binds to a different target or a different epitope on TLR9.

The term “effective amount” as used herein, refers to that amount of TLR9 antagonist, which is sufficient to inhibit the progression of fibrosis in a subject when administered to a subject. An effective amount will vary depending upon the subject and the severity of the fibrotic disorder, the weight and age of the subject, the manner of administration and the like, which can readily be determined by one of ordinary skill in the art. TLR9 antagonist dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, of a TLR9 antagonist. Dosage regimens may be adjusted to provide the optimum therapeutic response. An effective amount is also one in which any toxic or detrimental effects (i.e., side effects) of a TLR9 antagonist are minimized and/or outweighed by the beneficial effects.

Actual dosage levels of the TLR9 antagonist used in the methods of the present invention may be varied so as to obtain an amount of the active ingredient which is effective to achieve the desired response, e.g., inhibiting the progression of fibrosis, for a particular patient, composition, and mode of administration, without being toxic to the patient. The selected dosage level will depend upon a variety of pharmacokinetic factors including the activity of the particular TLR9 antagonist employed, or the ester, salt or amide thereof, the route of administration, the time of administration, the rate of excretion of the particular antagonist being employed, the duration of the treatment, other drugs, compounds and/or materials used in combination with the particular antagonist employed, the age, sex, weight, condition, general health and prior medical history of the patient being treated, and like factors well known in the medical arts. A physician or veterinarian having ordinary skill in the art can readily determine and prescribe the effective amount of the antagonist required. For example, the physician or veterinarian could start doses of the antagonist at levels lower than that required in order to achieve the desired therapeutic effect and gradually increase the dosage until the desired effect is achieved. In general, a suitable daily dose of a TLR9 antagonist will be that amount which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a TLR9 antagonist may be administered as two, three, four, five, six or more sub-doses administered separately at appropriate intervals throughout the day, optionally, in unit dosage forms. While it is possible for a TLR9 antagonist of the present invention to be administered alone, it is preferable to administer the antagonist as a pharmaceutical formulation (composition).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the TLR9 antagonists used in the methods of the present invention may be administered once or twice weekly by subcutaneous injection or once or twice monthly by subcutaneous injection.

To administer a TLR9 antagonist used in the methods of the present invention by certain routes of administration, it may be necessary to include the antagonist in a formulation suitable for preventing its inactivation. For example, the TLR9 antagonist may be administered to a subject in an appropriate carrier, for example, liposomes, or a diluent. Pharmaceutically acceptable diluents include saline and aqueous buffer solutions. Liposomes include water-in-oil-in-water CGF emulsions, as well as conventional liposomes (Strejan et al. (1984) J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active TLR9 antagonist, use thereof in a pharmaceutical compositions is contemplated. Supplementary active compounds can also be incorporated with the TLR9 antagonist.

TLR9 antagonists used in the methods of the invention typically must be sterile and stable under the conditions of manufacture and storage. The antagonist can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including an agent that delays absorption, for example, monostearate salts and gelatin.

Sterile injectable solutions can be prepared by incorporating the active antagonist in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

TLR9 antagonists that can be used in the methods of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the antagonist which produces a therapeutic effect. Generally, out of one hundred per cent, this amount will range from about 0.001% to about 90% of active ingredient, preferably from about 0.005% to about 70%, most preferably from about 0.01% to about 30%.

The phrases “parenteral administration” and “administered parenterally”, as used herein, means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed along with the TLR9 antagonists utilized in the methods of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

TLR9 antagonists may also be administered with adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When TLR9 antagonists used in the methods of the present invention are administered to humans and animals, they can be given alone or as a pharmaceutical antagonist containing, for example, 0.001 to 90% (more preferably, 0.005 to 70%, such as 0.01 to 30%) of active ingredient in combination with a pharmaceutically acceptable carrier.

TLR9 antagonists can be administered with medical devices known in the art. For example, in a preferred embodiment, an antagonist can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medications through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

### III. Kits of the Invention

The invention also provides kits for prognosing the progression of fibrosis in a subject having fibrosis. These kits include means for determining the level of expression of TLR9 and instructions for use of the kit.

The kits of the invention may optionally comprise additional components useful for performing the methods of the invention. By way of example, the kits may comprise means for obtaining a biological sample from a subject, a control sample, e.g., a sample from a subject having slowly progressing fibrosis and/or a subject not having fibrosis, one or more sample compartments, an instructional material which describes performance of a method of the invention and tissue specific controls/standards.

The means for determining the expression level of TLR9 can include, for example, buffers or other reagents for use in an assay for evaluating expression (e.g., at either the mRNA or protein level). The instructions can be, for example, printed instructions for performing the assay for evaluating the level of expression of TLR9.

The means for isolating a biological sample from a subject can comprise one or more reagents that can be used to obtain a fluid or tissue from a subject, such as means for obtaining a bronchial lavage or a transbronchial biopsy. The means for obtaining a biological sample from a subject may also comprise means for isolating peripheral blood mononuclear cells from a blood sample, for example by positive selection of the monocytes or by negative selection in which all other cell types other than monocytes are removed.

The kits of the invention may further comprise means for culturing a sample obtained from a subject.

The kits of the invention may also comprise means for determining the presence or absence of unmethylated CpG, means for determining the presence or absence of a gammaherpesvirus, the means for determining the level of expression of an additional marker selected from the group consisting of annexin 1, alpha smooth muscle actin, neutrophil elastase, KL-6, ST2, 1L-8, alpha defensin, beta3-endonexin, serine protease inhibitor, Kazal type, plasminogen activator inhibitor-1, HPS3, Rab38, Smad6, ADAMTS7, CXCR6, Bcl2-L-10, and MMP-9, and/or means for determining responsiveness of a cultured sample obtained from a subject to TGFβ and CpG. In one embodiment, the kits of the invention further comprise means for determining modulation of the expression and/or activity of alpha smooth muscle actin. In one embodiment, the means for determining modulation of the expression and/or activity of alpha smooth muscle actin includes means for determining responsiveness of a sample obtained from the subject to TGFβ and CpG.

In one embodiment, a kit of the invention includes means for obtaining a biological sample from a subject, e.g., a transbronchial biopsy, means for determining modulation of the expression and/or activity of alpha smooth muscle actin (e.g., by determining responsiveness of the biological sample obtained from a subject to TGFβ and CpG), and instructions for use of the kit. In one embodiment, such kits may further comprise determining the level of expression of TLR9. In another embodiment, such kits do not include means for determining the level of expression of TLR9.

Preferably, the kits are designed for use with a human subject.

The present invention is further illustrated by the following examples which should not be construed as further limiting. The contents of all references, patents and published patent applications cited throughout this application, as well as the Figures, are expressly incorporated herein by reference in their entirety.

### EXAMPLES

### I. Materials and Methodologies

In this section, the materials and methodologies used in the Examples are described.

### Mice.

All procedures described below were performed in a sterile, laminar environment and were approved by an animal care and use committee. Adult aged-matched, female C.B-17-scid-beige (C.B-17SCID/bg) mice (Taconic Farms, Germantown, N.Y.) were used. SCID mice were housed in a separate SPF (specific pathogen-free) facility for immunocompromised mice. C.B-17SCID/bg mice have two mutations: the first is the scid mutation, and the second is a beige mutation leading to a major defect in cytotoxic T-cell and macrophage function and a selective impairment in NK cell function.

### Human-SCID Model of AE-IPF.

Single-cell preparations of IPF/UIP (from clinically-classified rapid or slow progressors) and normal fibroblasts were obtained after trypsinization of 150-cm² tissue culture flasks and labeled with PKH26 dye according to the manufacturer's directions (Sigma Co., St. Louis, Mo.). Each labeled fibroblast line was diluted to 2×10⁶ cells/mL of phosphate- buffered saline (PBS), and 0.5 ml of this suspension was injected via a tail vain into groups of five to ten SCID mice. Thirty-five days later, all groups of mice were mildly anesthetized and received a single bolus of CpG-ODN (dissolved in sterile saline) or saline by intranasal delivery. Mice were euthanized by cervical dislocation 63 days after the i.v. human pulmonary fibroblast transfer. Whole-lung tissue was dissected at these times for histological and biochemical analysis (see below).

### IPF Patients.

Twenty-three patients diagnosed with IPF using a multidisciplinary, clinical/radiological/pathological mechanism were analyzed (Flaherty, K. R., et al. (2004) Am J Respir Crit Care Med 170:904-910). Baseline data included detailed clinical assessment, physiological studies, high resolution computed tomography (HRCT), and surgical lung biopsies (SLBs). Semi-quantitative scores of HRCT abnormality were generated using validated methodology (Kazerooni, E.A., et al. (1997) AJR Am J Roentgenol 169:977-9 83). Patients were treated with a variety of treatment regimens and followed closely with physiological studies and capture of clinical information during acute events. Using methodology that has been validated disease progression during the first year of follow-up utilized a composite of physiological deterioration (Flaherty, K. R., et al. (2006) Am J Respir Crit Care Med 174:803-809). The physiological criteria include an FVC decrease >10% and a DLCO decrease >15% based on baseline physiological abnormality. Acute exacerbations of IPF were defined using criteria (Collard, H. R., et al. (2007) Am J Respir Crit Care Med 176:636-643) or all-cause mortality This composite approach is now common in NHLBI (ACE IPF) and industry sponsored trials (BUILD 3, Artemis) (www. Clinicaltrials.gov).

### Cell Culture and Monocyte Differentiation Assay.

Blood was collected from healthy adult volunteers. PBMCs were isolated from EDTA blood by Ficoll-Paque Plus (GE Healthcare Biosciences, Piscataway, N.J., USA) according to the manufacturer's instructions. CD14+ monocytes were purified by negative selection using the Human Monocyte Isolation Kit II and MACS® LS column separators (Miltenyi Biotec). Briefly, a cocktail of biotin-conjugated antibodies against CD3, CD7, CDI6, CD19, CD56, CD123, and CD235a (Glycophorin A), as well as anti-Biotin MicroBeads, yields highly pure unlabeled monocytes obtained by depletion of the magnetically labeled cells. CD 14+ monocytes (>97% pure as detected by FACS) analysis were plated at a density of 2.5×10⁶ cells/well in a 6-well plate containing EX-CELL® Hybri-Max™ protein-free medium (Sigma-Aldrich) plus 0.5% sterile BSA with or without 10 ng/mL TGFβ. After 3 days, monocytes were either unstimulated or restimulated with 50 μg/mL sterile CpG-ODN, non-CpG, or poly IC. Twenty-four hours later, monocyte cultures were visualized under phase-contrast microscopy or processed for FACS analysis as described. For gene expression analysis, TriZol® reagent was added to each well and RNA extraction was performed according to the manufacturer's instructions. RNA was purified using the RNAeasy RNA cleanup kit (Quiagen) and subjected to on column DNAase digestion (Quiagen). RNA concentration and purity was determined by Nanodrop and confirmed by agarose gel electrophoresis. Purified RNA was subsequently reverse-transcribed into cDNA by rtPCR and similar treatments were pooled for analysis.

### A549 Cell Culture and EMT Assay.

A549 cells were seeded at a concentration of 40,000 cells/well in 12-well culture plates containing DMEM supplemented with 10% fetal bovine serum, 100 U/mL penicitlin and 100 μg/mL streptomycin. Treatments consisted of media alone, CpG (at 5, 10, 50, 100, or 200 μg/mL) or TGFβ (0.1, 0.5, 1, 5,10 ng/mL). Cells were treated for 72 or 96 hours (as indicated) and then trypsinized for analysis as described.

siRNA Knockdown of TLR9.

A549 cells were seeded at a concentration of 10,000 cells/well in 12-well culture plates containing DMEM supplemented with 5% fetal bovine serum. Twenty-four hours later, cells remained untreated or treated with 50 nM ON-TARGETpIus non-targeting siRNA Pool, 50 nM ON-TARGETpIus Cyclophilin B Control siRNA Pool, or 50 nM TLR9 ON-TARGETpIus siRNA SMARTpool (Dharmacon, Thermo Scientific) in DharmaFECT transfection reagent according to the manufacturer's instructions. Cells were incubated for 48 hrs for RNA analysis or 96 hrs for protein analysis to confirm TLR9 knockdown. For CpG-mediated EMT, CpG at the indicated concentration (s) was added to the siRNA-treated cells for 72 or 96 hrs (as indicated) and then trypsinized for analysis as described.

### Statistical Analysis.

All results are expressed as mean±SEM or median as appropriate. Baseline characteristics of patients were contrasted by unpaired t-tests or Mann Whitney tests, as appropriate. Overall survival characteristics were contrasted between patients experiencing disease progression during the first year of follow-up compared to those that did not using Cox regression analysis. The means between groups at different time points were compared by two-way analysis of variance (Ivanova, L., et al. (2008) Am J Physiol Renal Physiol 294:F1238- 1248). Individual differences were further analyzed using the unpaired t-test with Tukey-Kramer multiple comparisons test where indicated. Values of P<0.1 (*), P<0.01 (**), and P<0.001 (***) were considered significant.

### Histological Analysis of Human-SCID Model of IPF.

After cervical dislocation, the right lobes from each mouse were dissected, fully inflated with 10% formalin solution, and placed in fresh formalin for 24 hours. Standard histological techniques were used to paraffin-embed each lobe, and 5-μm sections were stained with Masson's trichrome for histological analysis.

### Isolation and Culture of Primary Pulmonary Fibroblast Lines.

UIP (from clinically-classified rapid or stable progressors) and normal SLBs were finely minced and the dispersed tissue pieces were placed into 150-cm2 cell culture flasks containing DMEM supplemented with 15% fetal bovine serum, 1 mmol/L glutamine, 100 U/ml penicillin and 100 μg/ml streptomycin. All primary lung cell lines were maintained in DMEM-15 at 37° C. in a 5% CO2 incubator and were serially passaged a total of five times to yield pure populations of lung fibroblasts as previously described in detail (Hogaboam, et al. 2005 Methods Mol Med. 117:209-21). All primary fibroblast cell lines from each patient group were used at passages 6 to 10 in the experiments outlined below and all of the experiments were performed under comparable conditions. Each well in a six-well tissue culture plate was seeded with 2.5×105 fibroblasts and at the 75% confluence were stimulated for 24 hours with media alone or 10 ng/ml of human recombinant IL-4 with or without 100 mM of CpG-ODN (Cell Sciences, CA), a synthetic agonist of TLR9. Twenty-four hours later, cell-free supernatants were collected for analysis.

Preparation of RNA and cDNA from SLBs and Primary Pulmonary Fibroblast Lines.

After treatments as describe above, TriZol Reagent (Invitrogen Life Technologies, Carlsbad, Calif.) was added to each well and total RNA was then prepared according to the manufacturer's instructions. The same process was applied to seven (upper and lower lobes) rapid IPF/UIP, seven (upper and lower lobes) stable IPF/UIP and seven normal SLBs after they were thawed on ice. Purified RNA from SLBs and the fibroblasts was subsequently reversetranscribed into cDNA using a BRL reverse transcription kit and oligo (dT) 12-18 primers. The amplification buffer contained 50 mmol/L KCl, 10 mmol/L Tris-HCl, pH 8.3, and 2.5 mmol/L MgCl2 (Invitrogen Life Technologies, Carlsbad, Calif.).

### Real-Time TaqMan PCR Analysis.

Human TLR9, collagen 1, and asma gene expression was analyzed by a real-time quantitative RT-PCR procedure using an ABI PRISM 7500 Sequence Detection System (PE Applied Biosystems, Foster City, Calif.). The cDNAs from SLBs samples were analyzed for TLR9 and the cDNAs from cultured monocytes and A549 cells were analyzed for collagen 1 and αsma. GAPDH was used as an internal control. Primers and probe used for TLR9 were purchased from Applied Biosystems. The primers and probes used for collagen 1 were:

| (SEQ ID NO: 1) | |
|---|---|
| forward | TGGCCTCGGAGGAAACTTT |
| and | |
| (SEQ ID NO: 2) | |
| reverse | TCCGGTTGATTTCTCATCATAGC, |
| (SEQ ID NO: 3) | |
| MGB probe | CCCCAGCTGTCTTAT; |
| For αsma: | |
| (SEQ ID NO: 4) | |
| forward | GCGTGGCTATTCCTTCGTTACT |
| and | |
| SEQ ID NO: 5) | |
| Reverse | GCTACATAACACAGTTTCTCCTTGATG, |
| (SEQ ID NO: 6) | |
| MGB probe | TGAGCGTGAGATTGT. |

Gene expression was normalized to GAPDH, and the fold increases in targets gene expression was calculated as is indicated for each experiment.

### Hydroxyproline Assay.

Left lobe samples from each mouse were dissected, homogenized, and biochemically processed as described previously for the hydroxyproline assay (E S Chen, B M Greenlee, M Wills-Karp, D R Moller: Attenuation of lung inflammation and fibrosis in interferon-gamma-deficient mice after intratracheal bleomycin. Am J Respir Cell Mol Biol 2001, 24:545-55). Hydroxyproline concentrations were calculated from a hydroxyproline standard curve (0 to 100 μg of hydroxyproline/ml). The levels in each sample were normalized to the protein (in mg) present in each sample measured by the Bradford protein assay.

### Flow Cytometric Analysis.

Monocytes were incubated with Accutase™ (eBiosciences) for 15 minutes after a 4 days treatment to facilitate detachment from cell culture plates and subjected to a previously described protocol for flow cytometric analysis (D Pilling, T Fan, D Huang, B Kaul, R H Gomer: Identification of markers that distinguish monocyte-derived fibrocytes from monocytes, macrophages, and fibroblasts. PLoS One 2009, 4:e7475). Monocytes were stained with anti-CD14-PE-Cy7, anti-CD45RO-Pacific Blue, anti-CXCR4-FITC. For TLR9 and collagen staining, monocytes were permeabilized using BD Perm/Wash™ and stained with TLR9-PE and collagen-biotin labeled followed by strepavidin-APC. Cells were analyzed using a FACSCalibur and Cell Quest software (BD Biosciences, San Jose, Calif.).

### Immunofluorescence.

Monocytes were added to 8-well glass Labtek (Nunc inc., Naperville, Ill.) tissue culture plates at a cell density of 350,000 cells/well containing EX-CELL® Hybri-Max™ protein-free medium (Sigma-Aldrich) containing 0.5% sterile BSA and the indicated treatments for the specified experiment. A549 cells were added to 8-well glass Labtek plates at a density of 20,000 cells/well containing DMEM supplemented with 10% fetal bovine serum, 100 U/ml penicillin and 100 μg/ml streptomycin and the indicated treatments for the specified experiment. Cells were fixed with 4% paraformaldehyde and stained overnight at 4° C. with rabbit polyclonal anti-human collagen 1 (Abcam ab292) or rabbit IgG Isotype control (Abcam). After repeated washes in PBS, monocytes were

incubated with FITC-conjugated mouse anit-rabbit IgG for 1 h at 4° C. Cells were washed again in PBS, mounted, and visualized using a fluorescent microscope at 40× magnification.

### Example 1

### Clinical Features of Rapid Versus Slowly Progressive Forms of IPF and Identification of Differential TLR9 Expression in Surgical Lung Biopsies

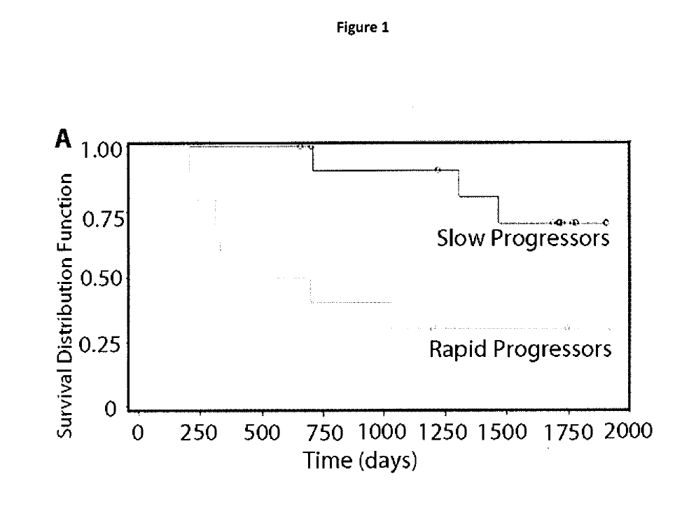

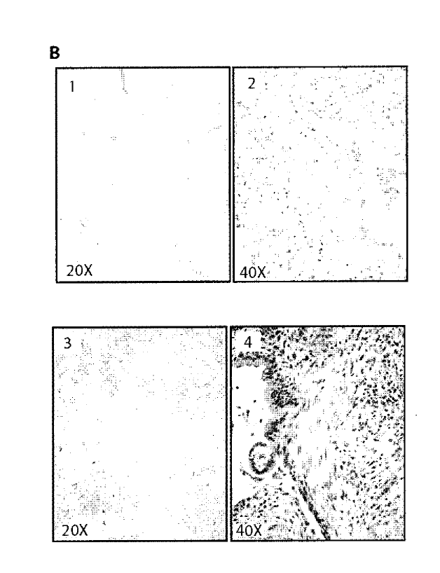

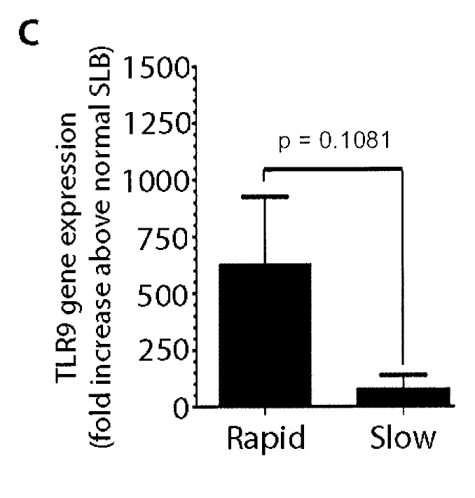

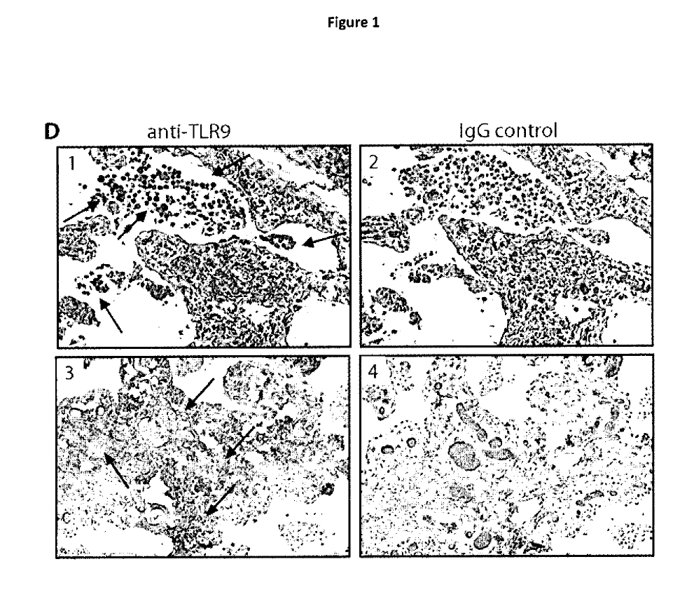

Ten IPF patients exhibited disease progression during the initial one year of follow-up while 13 did not; mean time of follow-up for the patients was 1154±03 days. Of the ten patients experiencing progressive disease during the first year of follow-up, eight were characterized as progressors based on physiological progression (FVC in 6, DLCO in 2), one experienced an acute exacerbation of IPF, and one died of respiratory causes over a time frame longer than used to define an acute exacerbation. Overall survival was better in patients who did not compared to those that did exhibit disease progression over the first year of follow-up (p=0.03) (FIG. 1A). Table 1 enumerates the clinical, physiological, imaging, and histological features at baseline.

TABLE 1
Clinical features of patients with rapid versus slowly progressive IPF.

| | Rapid Progressor | Slow Progressor | |
|---|---|---|---|
| Feature | (n = 10) | (n = 13) | P Value |
| Demographic | | | |
| Age | 64 ± 7 | 64 ± 5 | 0.9 |
| Gender (m/f) | 6/4 | 9/4 | 0.69 |
| Physiological | | | |
| FVC (% pred) | 63 ± 14 | 73.2 ± 17.5 | 0.17 |
| DLCO (% pred) | 44 ± 18 | 51 ± 26 | 0.09 |
| HRCT | | | |
| Alveolar | 1.53 ± 0.79 | 1.60 ± 0.71 | 0.85 |
| Interstitial | 1.31 ± 0.60 | 0.97 ± 0.38 | 0.11 |
| Histological | | | |
| HC score (median) | 1.1 | 1.0 | 0.55 |
| Max. HC score | 2.0 | 2.0 | 0.54 |
| (median) | | | |
| Disease | | | |
| progression* | | | |
| FVC > 10% | 6 | NA | NA |
| DLCO > 15% | 2 | NA | NA |
| AE IPF | 1 | NA | NA |
| Death | 1 | NA | NA |
| FVC slope | −0.05 ± 0.06 | 0.01 ± 0.04 | 0.13 |
| DLCO slope | −0.14 ± 0.17 | 0.10 ± 0.27 | 0.14 |

*Time to first event during first year of follow-up

It is evident that no statistically significant difference was noted in demographics, physiological severity, or HRCT/histological semi-quantitative abnormality. FIG. 1B demonstrates representative histology for slow (panels 1 and 2) and rapid progressors (panels 3 and 4). In both types of patient, the surgical lung biopsies demonstrated heterogeneous interstitial fibrosis with architectural distortion (panels 1 and 3) and multifocal fibroblast foci (panels 2 and 4) characteristic of UIP. No case had evidence of acute exacerbation of IPF (i.e. diffuse alveolar damage) at the time of surgical lung biopsy.

It has recently been reported that that TLR9 is highly expressed in IPF lungs and CpG-ODN drives myofibroblast differentiation of IPF lung fibroblasts in vitro (Meneghin, A., et al. (2008) Histochem Cell Biol 130:979-992). To test whether TLR9 expression differs in rapidly progressive IPF, TLR9 expression was quantitated in surgical lung biopsies from IPF patients clinically classified as rapid or stable progressors. FIG. 1C demonstrates that TLR9 gene expression is elevated in lungs from rapidly progressive IPF patients compared to normal and stable progressors. These results are confirmed by immunohistochemical analysis of TLR9 in surgical lung biopsies from rapid and slow progressors that demonstrates both intensity and localization of TLR9 protein (FIG. 1D). FIG. 1 D demonstrates increased TLR9 protein in the interstitial areas of the lung of rapid progressors (FIG. 1D panel 3) compared to slow progressors in which pronounced TLR9 staining is appears to be demonstrated by the immune cells (FIG. 1D panel 1).

### Example 2

### CpG-ODN Induces a Fibroblast-Like Phenotype in Primary Human Blood Monocytes In Vitro in the Presence of TGFβ

Based on previous findings that CpG induces myofibroblast differentiation of IPF fibroblasts, it was determined whether CpG can also drive a fibroblast-like phenotype in other cell types relevant to the pathogenesis of IPF. The effects of CpG effects on the human blood monocytes, which are central facilitators of immunological responses to invading pathogens was tested. Separate studies have previously reported that fibroblast-like cells (“fibrocytes”) can arise from purified human CD14+ monocytes under serum-free conditions within 4 days (Pilling, D., et al. (2003) J Immunol 171:5537-5546; Shao, D.D., et al. (2008) J Leukoc Biol 83:1323-1333; Hong, K. M., et al. (2007) J Biol Chem 282:22910-22920). This in contrast to other reports demonstrating a fibrocyte population devoid of CD 14 in human PBMC cultures after 7 days in the presence of serum (Hong, K. M., et al. (2007) J Biol Chem 282:22910-22920; Yang, L., et al. (2002) Lab Invest 82:1183-1192). In these studies, the addition of TGFβ to PBMC cultures promoted fibrocyte differentiation, which is minimally defined by spindle-shaped morphology and collagen I expression. Thus, it was determined whether CpG can drive a fibrocyte-like phenotype in purified CD14+ monocytes.

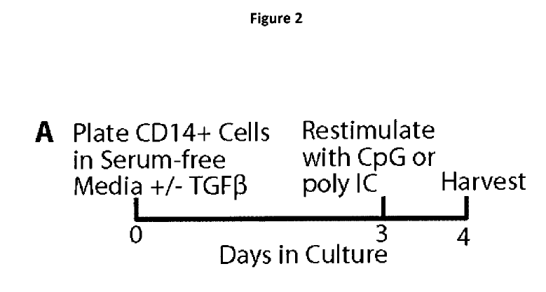

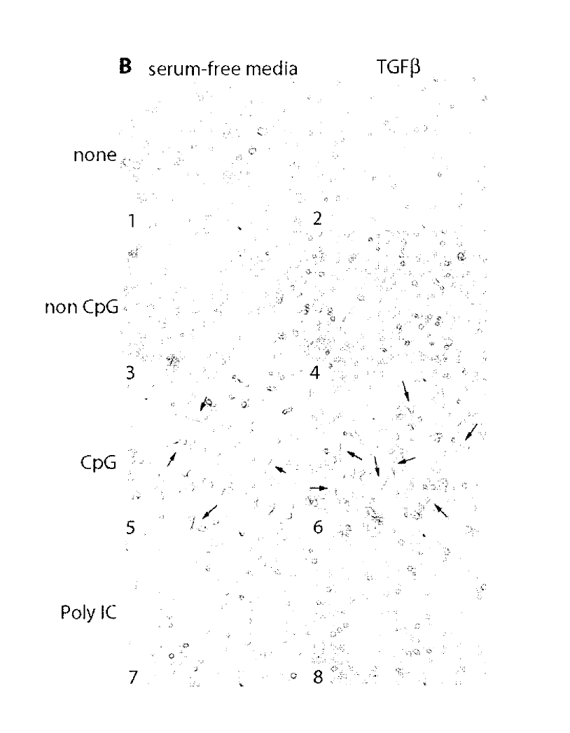

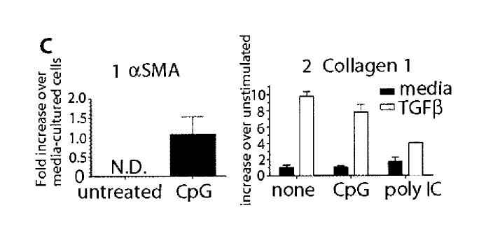

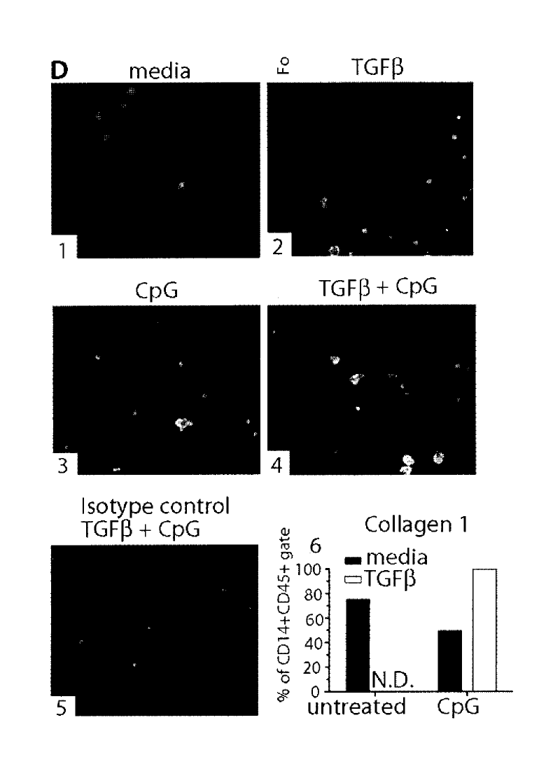

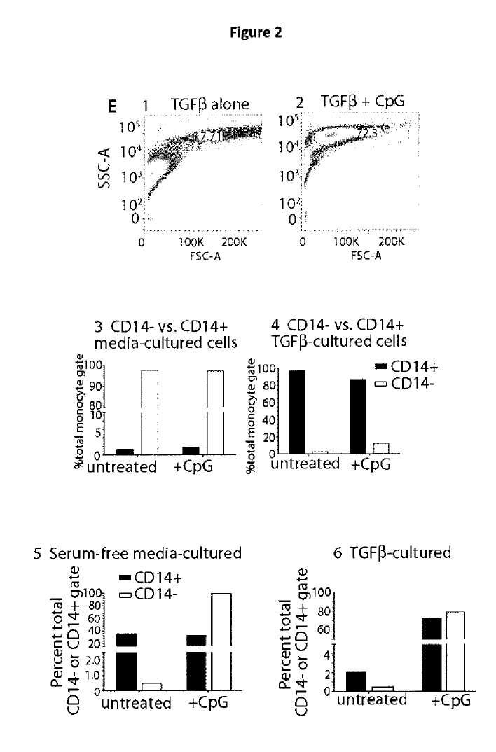

Peripheral blood monocytes from healthy human donors were purified for CD14-expressing cells by negative selection that depleted T and B cells, dendritic cells, NK cells, erythrocytes, and stem cells. FIG. 2A indicates that purified CD14+ cells were plated in serum-free media in the presence or absence of 10 ng/mL TGFβ for 3 days, after which they were stimulated for an additional day with nothing, control nonstimulatory CpG-ODN (non CpG), CpG ODN, or a TLR3 agonist (Poly I-C). Morphological assessment by phase-contrast microscopy revealed that monocytes cultured in media alone or in combination with TGF maintained a rounded shape typical of a monocytic phenotype (FIG. 2B panels 1 and 2). The same phenotype was observed in macrophages stimulated with non-CpG (FIG. 2B panels 3 and 4) and poly I-C (FIG. 2B panels 7 and 8) both in the absence and presence of TGFβ. In contrast, monocytes stimulated with either CpG alone (FIG. 2B panel 5) and/or with CpG in the presence of TGFβ (FIG. 2B panel 6) exhibited a distinct population of elongated, spindle-shaped cells resembling fibrocytes.

To determine whether the differences observed in the cultures corresponded with the induction of fibrocyte markers, RNA was isolated and purified from the adherent cells and subjected to gene expression analysis by quantitative TaqMan real-time PCR. Alpha smooth muscle actin (αSMA) is a specific protein marker expressed primarily on mesenchymal cells such as smooth muscle and fibroblasts, and is typically absent in non-structural cells. Upregulation has been linked to myofibroblast differentiation and, more recently, fibrocyte differentiation. Induction of αSMA gene transcript was only observed in monocytes stimulated with CpG (FIG. 2C panel 1). TGFβ did not alter αSMA gene expression in cells that were stimulated with CpG, indicating that upregulation of αSMA gene expression in the culture system is specific to CpG.

It was observed that, in agreement with previous studies, monocytes demonstrate upregulation of collagen 1 when cultured in the presence of TGFβ (FIG. 2C panel 2). Interestingly, however was the observation that unstimulated monocytes also express collagen, which is consistent with previous reports that have reported that macrophages indeed express the entire repertoire of collagens (Schnoor, M., et al. (2008). J Immunol 180: 5707-5719). Though no differences in collagen expression were observed which correlated with the previous morphological differences that were observed (i.e. CpG-induced elongated, spindle-shaped cells), these data confirm that TGFβ increases collagen expression in CD14+ monocytes but that this effect may only be limited to TGFβ.

It was next determined whether CpG affects collagen protein expression in cultured CD14₊ monocytes by immunohistochemistry. FIG. 2D demonstrates specific upregulation of collagen staining in CD14+ monocytes that were cultured with either TGF alone (panel 2) and stimulated with CpG alone in media (panel 3). Treatment with both CpG and TGF enhance collagen 1 staining (panel 4), which is consistent with the change in morphology demonstrated in FIG. 2B panel 6. Furthermore, flow cytometry quantification of collagen-positive CD14+CD45+ monocytes indicates that CpG enhances collagen 1 protein expression in TGFβ-cultured cells (panel 6).

The fibrocyte-like monocytes were characterized using flow cytometric analysis. Initial observations of forward and side scatter properties of CD14+ monocytes cultured in presence or absence of TGFβ confirmed that CpG induces morphological changes that are consistent with a fibroblast-like cell shape. FIG. 2E (panel 1) demonstrates that the majority of monocytes cultured in TGFβ alone appear smaller in size (panel 1). In contrast, monocytes stimulated with CpG in the presence of TGFβ have a dominant population (72.3% of total cells) comprised of cells having increased forward scatter and side scatter, indicative of increased cellular size and complexity (panel 2).

Monocyte-derived fibrocytes are widely characterized as CD14-negative, and other groups have demonstrated that PBMCs lose CD14 expression upon differentiation into fibrocytes (Abe, R., et al. (2001) J Immunol 166:7556-7562; Gomperts, B. N. & Strieter, R. M. (2007) J Leukoc Blol 82:449-456). Moreover, CD14 is a cell surface co-receptor for LPS, along with TLR4 and MD-2, on macrophages that can be shed during bacterial infections (Moreno, C., et al. (2004) Microbes Infect 6:990-995: Sandanger, O., et al. (2009) J Immunol 182:588-595). It was determined whether the presence or absence of TGFβ affects the CD14+ monocyte population during fibrocyte differentiation in this culture system described herein. Panel 3 in FIG. 2E demonstrated that after 4 days, >95% of the total cells are CD14− when cultured in media alone, and CpG does not affect the population. Opposite results are shown in panel 4, in which CD 14+ monocytes comprise almost 100% of the cell population when cultured in media containing TGFβ or TGFβ and CpG. These results demonstrate that CD 14 expression on monocytes is dynamic, and that loss or maintenance of CD 14 expression does not necessarily correlate with their fibrocytes differentiation.

The effects of CpG on the CD14+ and CD14− monocyte population for upregulation of established fibrocytes markers was determined by flow cytometry. It was found that in CD14− cells CpG alone or in combination with TGFβ induces expression of CD45, a hematopoietic marker widely used to characterize fibrocytes (FIG. 2E panels 5 and 6). Upregulation of CD45 by CpG was also observed in CD 14+ monocytes that were cultured with TGFβ (FIG. 2E panel 6). No effect on CD45 expression was observed in CD14+ cells cultured in media alone (FIG. 2E panel 5). Collectively, these data indicate that CpG induces a fibrocyte-like phenotype in CD 14+ monocytes defined by induction of an elongated, spindle-shaped morphology, and upregulation of αSMA, collagen 1 and CD45 protein.

### Example 3

### CpG-ODN Induces Epithelial-Mesenchymal Transition in A549 Cells

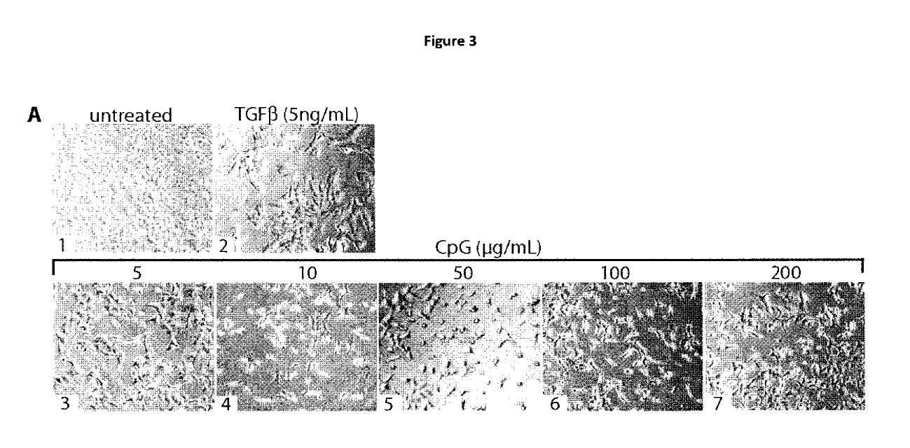

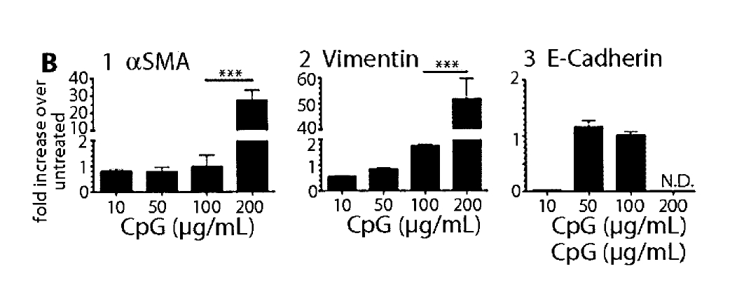

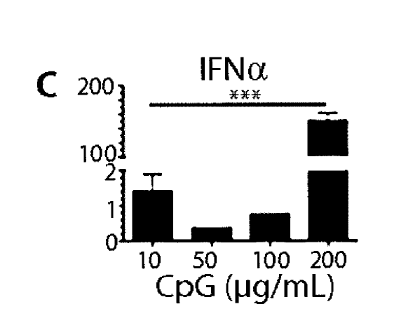

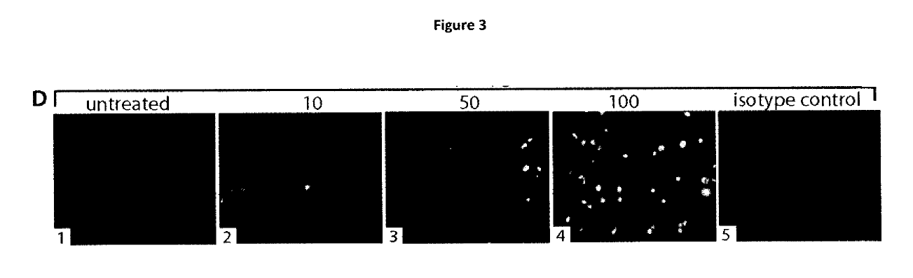

Based on the CpG effects observed in monocytes (FIG. 2), it was postulated that CpG may induce a classic EMT response in epithelial cells. The human adenocarcinoma type II alveolar epithelial cell line, A549, has been widely used to investigate TGFβ-driven EMT (Rho, J. K., et al. (2009). Lung Cancer 63:21 9-226; Illman, S. A., et al. (2006) J Cell Sci 119, 3856-3865; Kasai, H., et al. (2005) Respir Res 6:56). Treatment of A549 with TGFβ results in cell spreading and elongation, loss of epithelial cell markers such as E-cadherin, and expression of mesenchymal proteins including αSMA, collagen 1, and Vimentin. Untreated A549 cells after 96 hours in culture media maintained a cobblestone epithelial morphology and growth pattern (FIG. 3A panel 1). As a positive control, A549 cells were treated with increasing concentrations of TGFβ and observed obvious morphological changes with as little as 0.1 ng/mL. FIG. 3A panel b is a representative image of A549 cells cultured with 5 ng/mL for 96 hours and demonstrates TGFβ-induced cell spreading and a fibroblast-like morphology. To test whether CpG can also induce these changes, A549 cells were treated with increasing concentrations of CpG for 24, 48, 72, and 96 hours and assessed morphological changes and expression of EMT markers. FIG. 3A panels 3-7 demonstrate that CpG treatment induces cell spreading and elongated, spindle-shaped cells in a concentration-dependent manner during a 96-hour treatment.

Changes in cell morphology assessed under phase contrast light microscopy were observed as early 24 hours with the lowest concentration of CpG, however the most dramatic effects occurred after 72 and 96 hours. To confirm whether the morphological changes observed with CpG corresponded with EMT, RNA was isolated from the cultured A549 cells and gene expression of EMT markers was measured. FIG. 3B shows that CpG stimulates expression of αSMA, with an optimal effect at 200 μg/mL CpG (panel 1) and expression. CpG treatment of A549 cells also results in a concentration-dependent induction of Vimentin with an optimal effect at 200 μg/mL CpG (FIG. 3B panel 2) that is also accompanied by a loss of E-cadherin expression (FIG. 3B panel 3). In addition, fluorescent immunocytochemistry revealed a dose-dependent induction of collagen 1 by CpG in A549 cells after 96 hours (FIG. 3D panels 1-4). These data show that CpG induces EMT in lung epithelial cells. To determine whether CpG can also induce an innate immune response from A549 cells (Ronni, T., et al. (1997) J Immunol 158:2363-2374), IFNα gene expression was measured after increasing concentrations of CpG. Optimal IFNα gene transcription was detected in cells treated with 200 μg/mL (FIG. 3D), indicating that the EMT effects observed at this concentration also correlate with an innate immune response.

To determine whether CpG-DNA induction of EMT in A549 cells was TLR9 dependent, TLR9 protein expression was targeted by RNA interference and knockdown was assessed before testing CpG-mediated EMT in these cells. A549 cells treated with an siRNA pool consisting of 4 different specific sequences against nothing (non target), the reference protein cyclophillin B, or TLR9 were lysed 96 hours after a 96 hours treatment. FIG. 3E panels 1-4 confirm that TLR9 protein expression is ablated in cells treated with TLR9 siRNA but not non-target or cyclophilin B siRNA. Moreover, A549 cells at this timepoint appeared as those cultured in treatment media+transfection reagent alone (FIG. 3E panel 5) and no indication of stress response or changes in morphology were observed microscopically in cells cultured with non-target siRNA (FIG. 3E panel 6), cyclophilin B siRNA, or TLR9 siRNA (FIG. 3E panel 7). After TLR9 protein silencing was confirmed by Western Blot (FIG. 3E panels 1-4) in one of the triplicate wells from the same experiment, siRNAtreated A549 cells in the remaining duplicate wells were stimulated with CpGDNA for additional 72 hours and monitored throughout for changes in morphology. The morphology of A549 cells cultured in treatment media+transfection reagent appeared unaltered (FIG. 3E panel 8). FIG. 3E panel 9 indicates that non target siRNA has no effect on inhibiting CpG-mediated EMT, as indicated by cell spreading and elongated, spindle-shaped cells. This effect was also observed in cells treated with cyclophilin B siRNA. In contrast, A549 cells treated with TLR9 siRNA failed to demonstrate similar morphological changes (FIG. 3E panel 10). These cells appeared stressed and apoptotic, which may indicate that complete ablation of TLR9 may drive alternative innate immune responses in alveolar epithelial cells in the presence of CpG-DNA. To further demonstrate that CpG induces EMT in a TLR9-dependent manner, RNA from the siRNA and CpG-treated cultured A549 cells was isolated and gene expression of EMT markers were measured. FIG. 3E panel 11 and 12 demonstrates that TLR9 silencing by siRNA inhibits CpG-mediated induction of Vimentin expression and downregulation of E-cadherin expression, respectively.

### Example 4

### TLR9 Expression and Response to CpG-ODN is Increased in Rapidly Progressive IPF

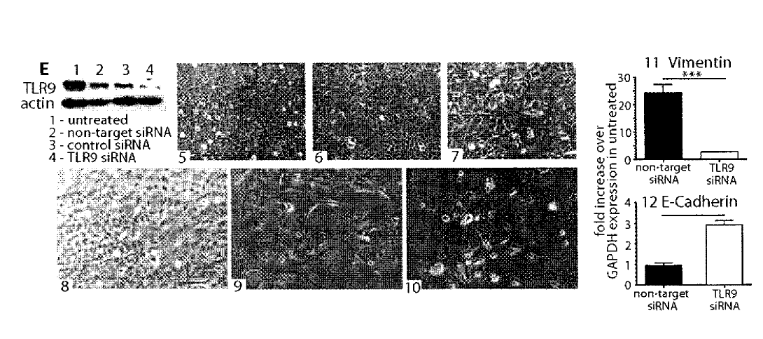

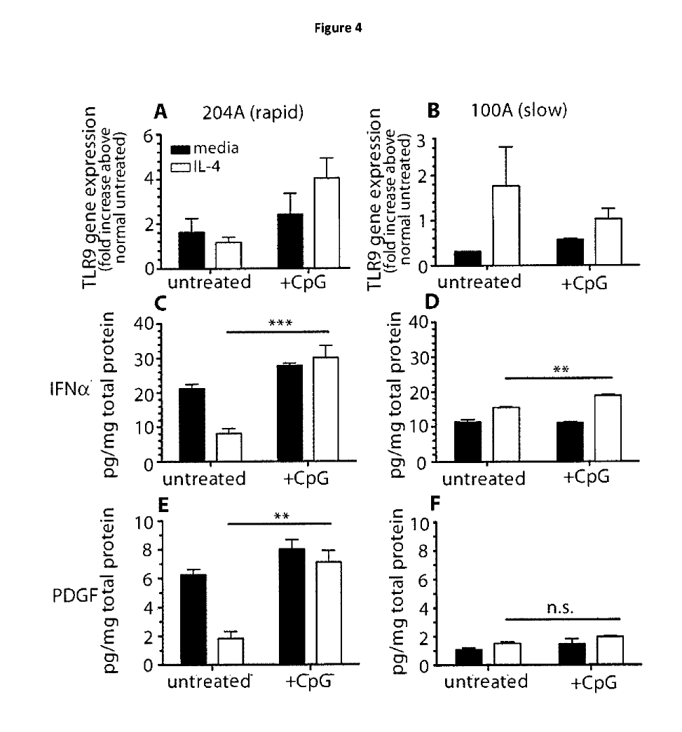

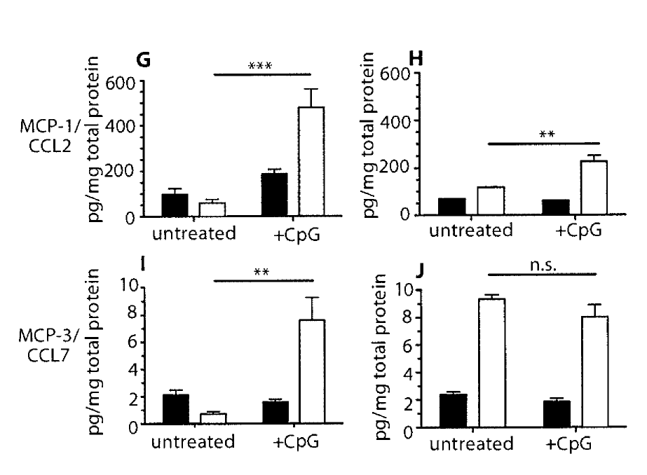

Representative lung fibroblasts from surgical lung biopsies obtained from patients exhibiting rapid disease progression were cultured in vitro with media alone or in the presence of a profibrotic stimulus, IL-4, to examine induction of TLR9 gene transcript. Stimulation of fibroblast cell line 204A (rapid progressor) with unmethylated CpG-ODN, TLR9 agonist, resulted in increased TLR9 expression (FIG. 4a) compared to that response observed with cell line 100A (slow progressor (FIG. 4b)).

In vitro cytokine production by rapid and slowly progressive fibroblasts was measured in cultured cell supernatants and compared in their responsiveness to CpG-ODN in the presence or absence of IL-4. Since type I interferons are secreted by cells upon effective TLR9 signaling, IFN-α protein levels were measured in supernatants from cultured fibroblast cell lines (Osawa, Y., et al. (2006) J Immunol 177:4841-4852). Rapidly progressive cell line 204A (FIG. 4c) demonstrates enhanced production of IFN-α compared to the slowly progressive line 100A (FIG. 4d) when stimulated with CpG in the presence of IL-4. This observation is consistent with the heightened expression of TLR9 by 204A the presence of both CpG-ODN and IL-4 (FIG. 4a). Rapidly progressive cell line 204A also demonstrates increased secretion of the profibrotic cytokines PDGF (FIG. 4e), MCP-I/CCL2 (FIG. 4g), and MCP-3/CCL3 (FIG. 2h) when stimulated with both CpG-ODN and IL-4. This is in contrast with the response observed with the slowly progressive line 100A, which does not show a comparable effect on the production of profibrotic cytokines with CpG in the presence of IL-4 (FIGS. 2f, 2h and 2j). Taken together, these data show a differential expression pattern of TLR9 and response to CpG between lung fibroblasts from rapid and slowly progressive IPF lungs.

### Example 5

### Rapidly Progressive Human IPF Fibroblasts Show Increased Fibrogenicity in a Humanized SCID Model of IPF

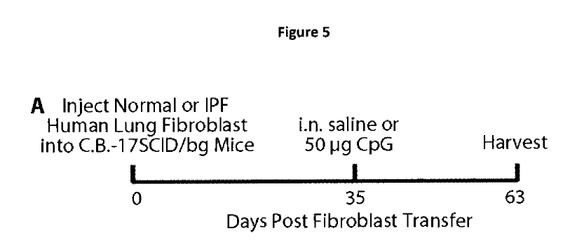

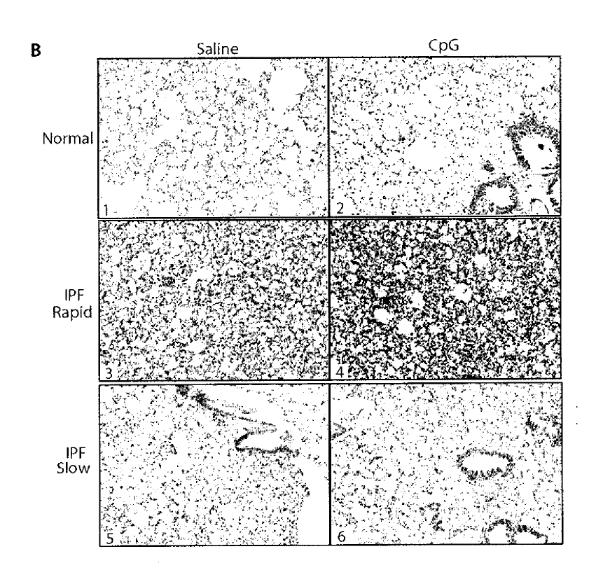

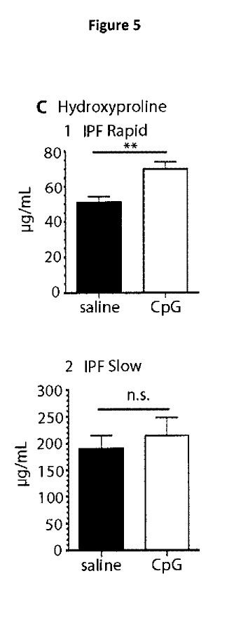

A previously described humanized SCID mouse model was used to test the fibrogenic potential of human lung fibroblasts from rapid versus slow progressors in vivo (Pierce, E. M., et al. (2007) Am J Pathol 170, 1152-1164). Representative lung fibroblasts cultured from rapid or slow progressors were previously analyzed in vitro (FIG. 4) and intravenously transferred into C.B.17SCID/bg mice. On Day 35 post transfer, mice were intranasally challenged with 50 μg CpG-ODN or saline and fibrosis was assessed on Day 63 post transfer (FIG. 5A). No pulmonary histopathology was observed in C.B.17SCID/bg mice that received normal pulmonary fibroblasts (FIG. 5B panel 1). Moreover, no effect was observed in these mice when challenged with CpG on Day 35 (FIG. 5B panel 2). Histological assessment of mouse lungs by Trichrome stain on Day 63 post transfer revealed that transfer of rapidly progressive human UIP/IPF fibroblasts demonstrated collagen deposition and apparent disruption of the alveolar space associated with severe interstitial thickening and remodeling (FIG. 5B panel 3). Furthermore, fibrosis was markedly enhanced in those lungs that received a CpG challenge on Day 35 (FIG. 5B panel 4). This is in striking contrast to the degree of fibrosis observed in mouse lungs that received stable human UIP/IPF fibroblasts and a CpG challenge. FIG. 5B demonstrates that stable UIP/IPF human lung fibroblasts cause a modest fibrotic response in mouse lungs as assessed on Day 63 post transfer (panel 5) which is not enhanced by a CpG stimulus (panel 6). Hydroxyproline is a commonly used marker of de novo collagen synthesis in experimental models of fibrosis. In this study, hydroxyproline levels were measured on Day 35 in half lung samples from C.B.17SCID/bg mice that had received UIP/IPF human fibroblasts from rapid progressors, and either challenged with saline or CpG on Day 35. As shown in FIG. 5C panel a, CpG challenge significantly increases hydroxyproline content only in mouse lungs transplanted with fibroblasts from rapidly progressive UIP/IPF patients, correlating with the histological assessment of increased collagen deposition in lungs from mice adoptively transferred with rapidly progressive UIP/IPF fibroblasts. Moreover, FIG. 5C panel 2 confirms the histology in FIG. 5B (panels 5 and 6: CpG challenge does not result in an increase in hydroxyproline content in mouse lungs transplanted with fibroblasts from slowly progressive UIP/IPF patients.

### Discussion

Idiopathic pulmonary fibrosis (IPF) is a chronic, generally progressive lung disease with high mortality and unmet clinical needs. There is growing evidence that, in addition to the proliferation of resident fibroblasts, these cells also arise from other cellular sources such as bone-marrow-derived fibrocytes and epithelial cells (Laurent, G. J., et al. (2005) Proc Am Thorac Soc 5:311-315). Several groups have demonstrated that fibrocytes enter the damaged tissue through chemokine dependent mechanism and mature into collagen-producing myofibroblasts (Mehrad, B., et al. (2007) Biochem Biophys Res Commun 353:104-108; Ishida, V., et al. Am J Pathol 170:843-854; Moore, B. B., et al. (2006) Am J Respir Cell Mol Biol 35:175-181; Kisseleva, T., et al. (2006) J Hepatol 45:429-438). Moreover, others have shown that epithelial structures differentiate into myofibroblasts though epithelial-mesenchymal transition (EMT) (Iwano, M., et al. (2002) J Clin Invest 110:341-350; Kim, K. K., et al. (2006) Proc Natl Acad Sci USA 103:13180-13185; Rygiel, K. A., et al. (2008) Lab Invest 88:112-123; Zeisberg, M., et al. (2007) J Biol Chem 282:23337-23347). These proposed mechanisms for the pathogenesis of fibrotic disease are common among all tissues such as the kidney, liver, skin, and lung. Further investigation of the pathways involved may improve the treatment of patients with variable forms of fibrotic diseases such as IPF.

Several hypotheses have been proposed for the etiology of IPF disease progression but still no consensus has been reached. Although therapeutic agents, such as anti-inflammatory drugs, are often used to treat fibrosis, such treatments can have undesirable side effects. Moreover, there are currently no wholly effective treatments or cures for fibrotic disorders.

Increasingly, it has become evident that the disease course in IPF patients is extremely variable with some patients exhibiting disease stability for prolonged periods of time while other exhibit rapid disease progression (Martinez, F. J., et al. (2005) Ann Intern Med 142:963-967). Although some IPF patients exhibit physiological decline others experience acute deterioration, acute exacerbation of IPF (AE-1PF) (Hyzy, R., et al. (2007) Chest 132, 1652-1658). As such, disease progression in IPF patients has been defined using a composite approach, which includes physiological progression, AE-IPF and/or all cause mortality. Rigorous studies aimed at understanding the etiology, risk factors, and pathogenesis of disease progression is required for the accurate treatment, prognosis, and predictors of IPF. The practical implication of this variability in disease progression is highlighted by the discordant results of two recently reported pirfenidone trials (Noble, P., et al. (2009) Am. J. Respir Crit Care Med. 179:A1129). In both studies, the pirfenidone-treated group exhibited a similar decrease in forced vital capacity percent predicted during follow-up (−6.49%) while the placebo group decreased by 9.55% in one study and 7.23% in the other. This difference resulted in vastly different results from the primary analyses (p=0.001 in one and p=0.501 in the second). As many current treatment studies emphasize approximately one-year term outcomes defining disease course during an initial evaluation would have great practical value.

AE-IPF remains poorly understood, and mortality of patients who present with this accelerated phase of the disease face death in period of weeks to a few months. Systematic studies of serum and BAL from patients with AE of IPF are lacking and, as such, no current molecular investigation of the pathogenesis of AE-IPF exists. Though the causes of AE-IPF are unknown, one possible explanation emerges from the detection of EBV in the lungs of IPF patients (Tsukamoto, K., et al. (2000) Thorax; Stewart, J. P., et al. (1999) Am J Respir Crit Care Med 159:1336-1341; Tang, Y. W., et al. (2003) J Clin Microbiol 41:633-2640): that an innate immune response to viral or bacterial infections may enhance the underlying fibrotic response. The present study strongly implicates the overexpression of TLR9, a pathogen recognition receptor, for driving rapid progression in IPF. In this study, the aim was to identify a mechanism of action by which the TLR9 accelerates the fibrotic process through its recognition of CpG DNA.

As described herein, surgical lung biopsies from rapidly progressive IPF patients clinically exhibit elevated levels of TLR9 gene transcript expression compared to those from stable IPF patients. Clinical data from these patients is described herein linking TLR9 expression to the rapid or slowly progressive phenotype of IPF. Those patients experiencing rapid clinical progression were similar to those exhibiting relative stability over the first year of follow-up with regards to demographic characteristics, physiological abnormality, semiquantitative radiological abnormality and pathological abnormality. Not surprisingly, patients exhibiting rapid progression exhibited overall worse survival compared to those with relative stability. Thus, the data shows that TLR9 is an indicator of IPF disease progression. Recently, annexin I was identified as a novel autoantigen present in patients with AE-IPF, however it was not addressed whether these patients also possessed a more robust measure of rapidly progressive disease (Kurosu, K., et al. (2008) J Immunol 181:756-767). Interestingly, this study reported that inflammatory infiltrates (primarily lymphocytes, neutrophils, and eosinophils) are elevated in the bronchoalveolar lavage of AE-IPF compared to that from stable IPF patients, which had undetectable amounts of these acute inflammatory cells. Elevations in neutrophil elastase, the mucin protein KL-6, ST2 protein, IL-8, and alpha defensin have also been previously reported in some patients with AE, suggesting a role for activated T cells and neutrophils (Mukae, H., at al. (2002) Thorax 57:623-62; Tajima, S., et al. (2003) Chest 124:1206-1214; Ziegenhagen, M. W., et al. (1998) Am J Respir Crit Care Med 157:762-768; Akira, M., et al. (1999) J Comput Assist Tomogr 23:941 -948; Yokoyama, A., et al. (1998) Am J Respir Crit Care Med 158:1680-1684). However, serum levels of these markers did not prove consistent predictors of prognosis (Shinoda, H., et al. (2009) Respiration).

To dissect a mechanism by which TLR9 may function as both a pathogenic sensor and as a profibrotic mediator in IPF, studies were conducted using peripheral blood monocytes from healthy donors. A recent report confirmed that circulating fibrocytes (defined as CD45+-ColI+) increase to an average of 15% of peripheral blood leukocytes in IPF patients who were evaluated during episodes of AE-IPF (Moeller, A., et al. (2009) Am J Respir Crit Care Med). The current study extends the examination of fibrocytes to identifying them as pathogenic sensors of CpG DNA. Since there was no access to blood monocytes from IPF patients in the midst of an acute exacerbation, naïve blood monocytes were utilized to investigate the agonistic potential of CpG in the context of fibrosis. Previous studies have demonstrated that bone marrow derived cells (fibrocytes) promote wound repair by migrating to wound sites and serving as a contributing source of myofibroblasts in fibrotic disease. Whether fibrocytes arise from monocytes remains controversial, though TGF has been shown to induce the in vitro differentiation of CD14+ monocytes into CD14−/collagen-1 fibrocytes. It has previously been demonstrated that CpG induces myofibroblast differentiation in cultured lung fibroblasts (Meneghin, A., et al. (2008) Histochem Cell Biol 130:979-992). Moreover, preliminary studies have indicated that CD14+ monocytes express significant levels of TLR9 gene transcript, which was in contrast to a previous report by Balmelli et al. that demonstrated expression of TLR7 but not TLR9 in fibrocytes (Balmelli, C., et al. (2007) Immunobiology 212:693-699). In the current study, the hypothesis that CpG may also induce the differentiation of CD 14+ monocytes into fibrocytes was tested. The data presented herein demonstrates that CpG treatment results in a hybrid monocyte phenotype, possessing both fibrocyte markers (spindle-shaped morphology, CD45, collagen 1, and α-sma expression) and CD14. It is also demonstrated herein that CpG enhances TGF differentiation, as demonstrated by increased cell size and increased immunostaining for collagen. These data confirm that monocytes can respond to CpG in a profibrotic manner, and may represent a separate cellular source for contributing to the myofibroblast population in the lung.

Consistent with these results, it is demonstrated herein that a CpG-mediated differentiation in the A549 human alveolar epithelial cell line that correlates with a myofibroblastic phenotype. It has been previously demonstrated that A549 cells express functionally active TLR9, and that CpG induces an antiapoptotic effect that may promote tumor progression (Droemann, D., et al. (2005) Respir Res 6:1). Though cytokine secretion was not measured in A549 cells herein, production of MCP-1/CCL2 in response to CpG may also lead to the attraction of immune cells (Droemann, D., et al. (2005) Respir Res 6:1). Although the effects of CpG reported herein are from a transformed cancer cell line and not in primary alveolar epithelial cells from IPF patients, the data show that alveolar epithelial cells from IPF lungs are less comparable to normal alveolar epithelial cells as evidenced by increased Wnt/ catenin signaling shown to drive epithelial cell injury, hyperplasia, and EMT in IPF lungs (Konigshoff, M., et al. (2008) PLoS ONE 3:e2142; Kim, K. K., et al. (2009) J Clin Invest 119:213-224). Indeed, it can be concluded from these data that CpG-DNA is recognized by TLR9 expressed on alveolar epithelial cells, promotes EMT, and is a candidate mechanism for the pathogenesis of AE-IPF.

Culturing lung fibroblasts from surgical lung biopsies from IPF patients has been instrumental for establishing a humanized mouse model of IPF (Pierce, E. M., et al. (2007) Am J Pathol 170, 1152-1164). In this study, this model was extended to investigate the role of TLR9 activation in progressive IPF. It was determined that lung fibroblasts from patients experiencing a rapidly progressive course demonstrate a hyperresponsiveness to CpG DNA challenge in a SCID model. A single bolus of CpG DNA given intranasally to mice transplanted with rapidly progressive IPF fibroblasts augmented the pulmonary fibrotic response in these mouse lungs, compared to those transplanted with normal or stable IPF fibroblasts. In vitro studies conducted with the same IPF fibroblast cell lines indicated that CpG stimulation results in the enhanced production of profibrotic cytokines from rapidly progressive fibroblasts. Therefore, in this SCID model, CpG induces the production of human profibrotic cytokines within the mouse lung and promotes an autocrine response from the human fibroblasts that results in increased fibrosis. These data show that CpG recognition by TLR9 within fibroblasts is another component of the mechanism by which bacterial or viral constituents augment fibrogenesis during progressive IPF.

TLR9 has recently been implicated in experimental models of other fibrosing diseases. Studies investigating the role of TLR9 in experimental liver fibrosis have demonstrated that TLR9-deficient mice show a protective fibrotic effect in the bile duct ligation (BDL) model of liver fibrosis, indicating a pathophysiological role for bacterial DNA and TLR9 in the development of hepatic fibrosis (Gabele, E., et al. (2008) Biochem Biophys Res Commun 376:271-276). CpG-ODN was also shown to increase renal fibrosis in a separate study using a murine model for lupus nephritis, as measured by the amount of interstitial fibroblast proliferation in MRL^{lpr/lpr} mice (Anders, H.J., et al. (2004) FASEB J 18:534-536). Moreover, other diseases, such as cancers, which result from aberrant cellular activation and proliferation, are susceptible to infectious exacerbations. CpG promotes cellular invasion in breast cancer epithelial cells as well as prostate cells in TLR9-mediated mechanism (Ilvesaro, J. M., et al. (2008) Mol Cancer Res 6:1534-1543; Ilvesaro, J. M., et al. (2007) Prostate 67:774-781; Merrell, M. A., et al. (2006) Mol Cancer Res 4:437-447). Chronic hepatitis C virus (HCV) infection that is associated with hepatocellular carcinoma has been recently shown to induce EMT in infected hepatocytes and promote cell invasion and metastasis (Battaglia, S., et al. (2009) PLoS ONE 4:e4355). In the current study it was tested whether alveolar epithelial cells respond to bacterial or viral components in a similar manner.

The clinical assessments utilized in the experiments described herein combined with in vitro and in vivo data acquired from IPF lung fibroblasts implicate TLR9 expression in the alveolar compartment to be an indicator of rapidly progressive IPF. It is demonstrated herein that expression of TLR9 on immune cells contributes to the exaggerated wound healing response that occurs in IPF patients exposed to a pathogenic stimulus. Therefore, measurement of TLR9 expression in surgical lung biopsies from routine diagnostic tests can be a predictive tool for determining whether IPF patients are susceptible to acute exacerbations and development of a rapidly progressive phenotype. The presence of bacterial DNA in serum and ascitic fluid is currently under active investigation as an indicator of poor prognosis in patients with liver cirrhosis (Zapater, P., et al. (2008) Hepatology 48:1924-1931; El-Naggar, M. M., et al. (2008) J Med Microbial 57:1533-1538). Though TLR9 was not evaluated in these studies, they provide rationale for measuring unmethylated CpG in serum and BAL from IPF patients, as well as TLR9 expression in IPF patient lung biopsies. Moreover, the current study provides impetus to investigate the therapeutic design of specific TLR9 antagonists. The addition of this diagnostic parameter can identify risk, improve the treatment protocol of IPF patients, and serve as a preventative approach for minimizing susceptibility to acute exacerbations.

### Example 6

### Primary Fibroblast Cultures Obtained from Subjects Having IPF May Be Used to Prognose Rapidly Progressive IPF

Transbronchial biopsies (approximately 20 mg of tissue) were isolated from subjects diagnosed as having IPF and cultured. Duplicate cultures from each primary fibroblast line were treated with either TGF□ or CpG. Results demonstrate that, regardless of clinical disease progression, all of the fibroblast cultures respond to TGF□, but only those fibroblasts from rapid progressors respond to CpG.

### EQUIVALENTS

Those skilled in the art will recognize, or be able to ascertain using no more than routine experimentation, many equivalents of the specific embodiments of the invention described herein. Such equivalents are intended to be encompassed by the following claims. Any combination of the embodiments disclosed in the dependent claims are contemplated to be within the scope of the invention.

1. A method for predicting the progression of fibrosis in a subject having fibrosis, the method comprising
determining the level of expression of Toll-like receptor 9 (TLR9) in a sample from the subject; and
comparing the level of expression of TLR9 in the sample from the subject to the level of expression of TLR9 in a control sample, wherein an increase in the level of expression of TLR9 in the sample from the subject as compared to the level of expression of TLR9 in the control sample is an indication that the fibrosis will rapidly progress,
thereby predicting the progression of fibrosis in the subject having fibrosis.

2. A method for identifying a compound that can slow down the progression of fibrosis in a subject having fibrosis, the method comprising:
separately contacting an aliquot of a sample from the subject with each member of a library of compounds;
determining the effect of a member of the library of compounds on the level of expression of Toll-like receptor 9 (TLR9) in each of the aliquots; and
selecting a member of the library of compounds which decreases the level of expression of TLR9 in an aliquot as compared to the level of expression of TLR9 in a control sample, thereby identifying a compound that can slow down the progression of fibrosis in a subject having fibrosis.

3. A method of monitoring the effectiveness of a therapy in reducing the progression of fibrosis in a subject having fibrosis, the method comprising
determining the level of expression of Toll-like receptor 9 (TL9R) in a sample from the subject prior to and following administration of at least a portion of the therapy to the subject; and
comparing the level of expression of TLR9 in the sample from the subject prior to the administration of the therapy to the level of expression of TLR9 in the sample from the subject following administration of at least a portion of the therapy, wherein a decrease in the level of expression of TLR9 in the sample following administration of at least a portion of the therapy as compared to the level of expression of TLR9 in the sample prior to the administration of the therapy is an indication that the subject is responding to the therapy, thereby monitoring the effectiveness of the therapy in reducing the progression of fibrosis in the subject having fibrosis.

4. A method of selecting a subject for participation in a clinical trial for a treatment of fibrosis, the method comprising
determining the level of expression of Toll-like receptor 9 (TLR9) in a sample from a subject having fibrosis, and
comparing the level of expression of TLR9 in the sample from the subject to the level of expression of TLR9 in a control sample, wherein a higher level of expression of TLR9 in the sample from the subject as compared to the level of expression of TLR9 in the control sample is an indication that the subject should participate in the clinical trial, thereby selecting a subject for participation in a clinical trial for a treatment of fibrosis.

5. The method of any one of claims 1-4, wherein the fibrosis is selected from the group consisting of idiopathic pulmonary fibrosis, liver fibrosis following liver transplantation, liver fibrosis following chronic hepatitis C virus infection, and interstitial fibrosis in focal segmental glomerulosclerosis.

6. The method of any one of claims 1-4, wherein the fibrosis is selected from the group consisting of cystic fibrosis of the pancreas and lungs, injection fibrosis, endomyocardial fibrosis, mediastinal fibrosis, myelofibrosis, retroperitoneal fibrosis, progressive massive fibrosis, nephrogenic systemic fibrosis.

7. The method of any one of claims 1-4, wherein the fibrosis is caused by surgical implantation of an artificial organ.

8.-23. (canceled)

24. A kit for predicting the progression of fibrosis in a subject having fibrosis, the kit comprising means for determining the level of expression of Toll-like receptor 9 (TLR9) and instructions for use of the kit to predict the progression of fibrosis in the subject having fibrosis.

25.-29. (canceled)

30. A method of inhibiting the progression of fibrosis in a cell, comprising contacting the cell with an effective amount of a Toll-like receptor 9 (TLR9) antagonist, thereby inhibiting progression of fibrosis in the cell.

31. The method of claim 30, wherein the cell is selected from the group consisting of a pulmonary cell, a liver cell, a kidney cell, a cardiac cell, a musculoskeletal cell, a skin cell, an eye cell, and a pancreatic cell.

32. A method for inhibiting the progression of fibrosis in a subject, comprising administering an effective amount of a Toll-like receptor 9 (TLR9) antagonist to the subject, thereby inhibiting the progression of fibrosis in the subject.

33. The method of claim 32, wherein the fibrosis is selected from the group consisting of idiopathic pulmonary fibrosis, liver fibrosis following liver transplantation, liver fibrosis following chronic hepatitis C virus infection, and interstitial fibrosis in focal segmental glomerulosclerosis.

34. The method of claim 32, wherein the fibrosis is selected from the group consisting of cystic fibrosis of the pancreas and lungs, injection fibrosis, endomyocardial fibrosis, mediastinal fibrosis, myelofibrosis, retroperitoneal fibrosis, progressive massive fibrosis, nephrogenic systemic fibrosis.

35. The method of claim 32, wherein the fibrosis is caused by surgical implantation of an artificial organ.

36.-38. (canceled)

39. The method of claim 30 or 32, wherein the TLR9 antagonist is selected from the group consisting of an antibody, a small molecule, a nucleic acid, a fusion protein, an adnectin, an aptamer, an anticalin, a lipocalin, and TLR9-derived peptidic compound.

40.-42. (canceled)

43. A method of monitoring the efficacy of a Toll-like receptor 9 (TLR9) antagonist to inhibit the progression of fibrosis in a subject, the method comprising
determining the level of expression of Toll-like receptor 9 (TLR9) in a sample from a subject who has been administered a TLR9 antagonist; and
comparing the level of expression of TLR9 in the sample from the subject to the level of expression of TLR9 in a control sample, wherein an increase in the level of expression of TLR9 in the sample from the subject as compared to the level of expression of TLR9 in the control sample is an indication that the TLR9 antagonist is not efficacious in inhibiting the progression of fibrosis in said subject and wherein a decrease in the level of expression of TLR9 in the sample from the subject as compared to the level of expression of TLR9 in the control sample is an indication that the TLR9 antagonist is efficacious in inhibiting the progression of fibrosis in said subject.


SEQUENCE LISTING (6 sequences)

SEQ ID NO: 1 - DNA, 19 bp, artificial sequence
  collagen forward primer
tggcctcggaggaaacttt

SEQ ID NO: 2 - DNA, 23 bp, artificial sequence
  collagen reverse primer
tccggttgatttctcatcatagc

SEQ ID NO: 3 - DNA, 15 bp, artificial sequence
  MGB probe
ccccagctgtcttat

SEQ ID NO: 4 - DNA, 22 bp, artificial sequence
  alpha sma forward primer
gcgtggctattccttcgttact

SEQ ID NO: 5 - DNA, 27 bp, artificial sequence
  alpha sma reverse primer
gctacataacacagtttctccttgatg

SEQ ID NO: 6 - DNA, 15 bp, artificial sequence
  MGB probe
tgagcgtgagattgt